(19) United States
(12) Patent Application Publication
(10) Pub. No.: US 2025/0387008 A1
(43) Pub. Date: Dec. 25, 2025

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Shumpei KAMON, Kanagawa (JP)

(73) Assignee: FUJIFILM Corporation, Tokyo (JP)

(21) Appl. No.: 19/315,701

(22) Filed: Sep. 1, 2025

Related U.S. Application Data
(63) Continuation of application No. PCT/JP2024/005563, filed on Feb. 16, 2024.

(30) Foreign Application Priority Data
Mar. 8, 2023 (JP) 2023-035948

(51) Int. Cl. A61B 1/00; A61B 1/05

(52) U.S. Cl. CPC A61B 1/000094; A61B 1/0005; A61B 1/05

(57) ABSTRACT

A medical support device includes a processor. The processor is configured to perform a measurement output process for measuring a size of an observation target region, based on a medical image obtained by imaging an imaging target region including the observation target region with a camera inserted into a body cavity and for outputting the size; and control the measurement output process in accordance with appearance information related to an appearance of a surface region in the medical image, the surface region being a portion of the imaging target region and including the observation target region.

### CROSS-REFERENCE TO RELATED APPLICATIONS

This application is a continuation application of International Application No. PCT/JP2024/005563, filed Feb. 16, 2024, the disclosure of which is incorporated herein by reference in its entirety. Further, this application claims priority from Japanese Patent Application No. 2023-035948, filed Mar. 8, 2023, the disclosure of which is incorporated herein by reference in its entirety.

### BACKGROUND

### 1. Technical Field

The technology of the present disclosure relates to a medical support device, an endoscope system, a medical support method, and a program.

### 2. Related Art

WO2019/078102A discloses a medical image processing device having an image acquisition unit, an image analysis processing unit, a notification execution unit, a notification setting information storage unit, and a notification setting information selection unit.

In the medical image processing device described in WO2019/078102A, the image acquisition unit acquires a medical image including a photographic subject. The image analysis processing unit performs image analysis processing of the medical image. The notification execution unit notifies a user of a result of the image analysis processing in accordance with notification setting information. The notification setting information storage unit stores the notification setting information in association with individual setting information different from the notification setting information. The notification setting information selection unit selects use notification setting information from the notification setting information stored in the notification setting information storage unit by using the individual setting information. The notification execution unit provides a notification in accordance with setting of the use notification setting information.

In the medical image processing device described in WO2019/078102A, the image analysis processing unit has a region-of-interest detection unit that detects a region of interest from the medical image as the image analysis processing, and a lesion determination unit that determines lesion information indicating the content of a lesion for the region of interest as the image analysis processing. The individual setting information is the lesion information. The lesion information is the size of the lesion or the type of the lesion.

JP2011-255006A discloses an image processing apparatus including an image acquisition unit, a region-of-interest detection unit, a determination unit, a display style control unit, and an alert image generation unit.

In the image processing apparatus described in JP2011-255006A, the image acquisition unit acquires an acquired image that is at least one of a normal-light image or a special-light image corresponding to the normal-light image, the normal-light image including a photographic subject image having information in a white wavelength range, the special-light image including a photographic subject image having information in a specific wavelength range. The region-of-interest detection unit detects a region of interest, which is a region where attention is focused, based on feature values of pixels in the acquired image. The determination unit determines whether to display an alert image corresponding to the region of interest in accordance with a result of detection of the region of interest. The display style control unit performs control to display an alert image corresponding to a display-target region of interest that is a region of interest for which the determination unit determines that the alert image is to be displayed.

In the image processing apparatus described in JP2011-255006A, the display style control unit performs control to display an alert image corresponding to the display-target region of interest from among alert images generated by an alert image generation unit and corresponding to regions of interest.

In the image processing apparatus described in JP2011-255006A, the determination unit determines whether to display an alert image generated by the alert image generation unit in accordance with a result of detection of the region of interest. The display style control unit performs control to hide an alert image determined to be hidden by the determination unit.

### SUMMARY

An embodiment according to the technology of the present disclosure provides a medical support device, an endoscope system, a medical support method, and a program that enable a user to grasp the size of an observation target region in a case where the appearance of a surface region including the observation target region in a medical image is appropriate.

A first aspect according to the technology of the present disclosure provides a medical support device including a processor, the processor being configured to perform a measurement output process for measuring a size of an observation target region, based on a medical image obtained by imaging an imaging target region including the observation target region with a camera inserted into a body cavity and for outputting the size, and control the measurement output process in accordance with appearance information related to an appearance of a surface region in the medical image, the surface region being a portion of the imaging target region and including the observation target region.

A second aspect according to the technology of the present disclosure is the medical support device according to the first aspect, in which positional relationship identification information enabling identification of a positional relationship is used as the appearance information, the positional relationship being a relative positional relationship between the camera and the surface region.

A third aspect according to the technology of the present disclosure is the medical support device according to the second aspect, in which a degree of luminance difference between a plurality of locations in the surface region in the medical image is used as the positional relationship identification information.

A fourth aspect according to the technology of the present disclosure is the medical support device according to the second aspect or the third aspect, in which a degree of frequency component difference between a plurality of locations in the surface region in the medical image is used as the positional relationship identification information.

A fifth aspect according to the technology of the present disclosure is the medical support device according to any one of the second to fourth aspects, in which a first depth of the surface region from the camera side is used as the positional relationship identification information, the first depth being obtained based on the medical image.

A sixth aspect according to the technology of the present disclosure is the medical support device according to any one of the second to fourth aspects, in which a second depth of the surface region from the camera side is used as the positional relationship identification information, the second depth being obtained using a depth sensor.

A seventh aspect according to the technology of the present disclosure is the medical support device according to any one of the second to sixth aspects, in which extension information related to an extension of the surface region in a depth direction from the camera side is used as the positional relationship identification information.

An eighth aspect according to the technology of the present disclosure is the medical support device according to any one of the second to seventh aspects, in which an angle between an optical axis of the camera and the surface region is used as the positional relationship identification information.

A ninth aspect according to the technology of the present disclosure is the medical support device according to any one of the second to eighth aspects, in which the processor is configured to perform no measurement of the size and/or no output of the size when the positional relationship is not a predetermined positional relationship, and perform the measurement output process when the positional relationship is the predetermined positional relationship.

A tenth aspect according to the technology of the present disclosure is the medical support device according to any one of the second to eighth aspects, in which the processor is configured to perform no measurement of the size and output first information when the positional relationship is not a predetermined positional relationship, the first information being information enabling identification of the size not being measured, and perform the measurement output process when the positional relationship is the predetermined positional relationship.

An eleventh aspect according to the technology of the present disclosure is the medical support device according to any one of the second to eighth aspects, in which the processor is configured to perform the measurement output process regardless of whether the positional relationship is a predetermined positional relationship, and output second information when the positional relationship is not the predetermined positional relationship, the second information being information enabling identification of the size being inaccurate.

A twelfth aspect according to the technology of the present disclosure is the medical support device according to any one of the second to eighth aspects, in which the processor is configured to perform the measurement output process regardless of whether the positional relationship is a predetermined positional relationship, and the measurement output process includes a perception level reduction process for reducing a level at which an output of the size in a case where the positional relationship is not the predetermined positional relationship is perceived such that the level becomes lower than a level at which an output of the size in a case where the positional relationship is the predetermined positional relationship is perceived.

A thirteenth aspect according to the technology of the present disclosure is the medical support device according to any one of the first to twelfth aspects, in which the observation target region of which the size is to be measured in the measurement output process is recognized from the medical image through an object recognition process performed on the medical image.

A fourteenth aspect according to the technology of the present disclosure is the medical support device according to any one of the first to thirteenth aspects, in which the observation target region of which the size is to be measured in the measurement output process is identified from the medical image in accordance with a provided instruction.

A fifteenth aspect according to the technology of the present disclosure is the medical support device according to any one of the first to fourteenth aspects, in which the size is output by displaying the size on a screen.

A sixteenth aspect according to the technology of the present disclosure is the medical support device according to any one of the first to fifteenth aspects, in which the medical image is an endoscopic image obtained by imaging the imaging target region with an endoscope.

A seventeenth aspect according to the technology of the present disclosure is the medical support device according to any one of the first to sixteenth aspects, in which the observation target region is a lesion.

An eighteenth aspect according to the technology of the present disclosure is an endoscope system including the medical support device according to any one of the first to seventeenth aspects and an endoscope provided with the camera.

A nineteenth aspect according to the technology of the present disclosure is a medical support method including performing a measurement output process for measuring a size of an observation target region, based on a medical image obtained by imaging an imaging target region including the observation target region with a camera inserted into a body cavity and for outputting the size; and controlling the measurement output process in accordance with appearance information related to an appearance of a surface region in the medical image, the surface region being a portion of the imaging target region and including the observation target region.

A twentieth aspect according to the technology of the present disclosure is the medical support method according to the nineteenth aspect, including using an endoscope provided with the camera.

A twenty-first aspect according to the technology of the present disclosure is a program for causing a computer to execute a medical support process including performing a measurement output process for measuring a size of an observation target region, based on a medical image obtained by imaging an imaging target region including the observation target region with a camera inserted into a body cavity and for outputting the size; and controlling the measurement output process in accordance with appearance information related to an appearance of a surface region in the medical image, the surface region being a portion of the imaging target region and including the observation target region.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example overall configuration of the endoscope system;

FIG. 3 is a block diagram illustrating an example hardware configuration of an electric system of the endoscope system;

FIG. 4 is a block diagram illustrating an example of functions of main components, according to an embodiment, of a processor included in a medical support device, and an example of information stored in an NVM;

FIG. 5 is a conceptual diagram illustrating an example of the content of a process of a recognition unit and a control unit;

FIG. 6 is a conceptual diagram illustrating an example of the content of a process of a control unit;

FIG. 7 is a conceptual diagram illustrating an example of the relative positional relationship between a surface region and a camera in a case where the appearance of a lesion in a frame is correct, and an example of the relative positional relationship between the surface region and the camera in a case where the appearance of the lesion in the frame is incorrect;

FIG. 8 is a conceptual diagram illustrating an example of the content of a process of a measurement unit;

FIG. 9 is a conceptual diagram illustrating an example of an aspect in which an endoscopic moving image and a size are displayed in a first display region and auxiliary information is displayed in a second display region;

FIG. 10 is a flowchart illustrating an example of the flow of a medical support process;

FIG. 11 is a conceptual diagram illustrating a first modification of display content displayed on a screen;

FIG. 12 is a conceptual diagram illustrating a second modification of display content displayed on the screen;

FIG. 13 is a conceptual diagram illustrating a third modification of display content displayed on the screen;

FIG. 14 is a conceptual diagram illustrating an example of the content of a process in which a distance difference is calculated by the control unit;

FIG. 15 is a conceptual diagram illustrating an example of the content of a process in which a luminance difference is calculated by the control unit;

FIG. 16 is a conceptual diagram illustrating an example of the content of a process in which a frequency component difference is calculated by the control unit;

FIG. 17 is a conceptual diagram illustrating an example of the content of a process in which a segmentation-corresponding region is identified by the control unit in accordance with an instruction received by a reception device;

FIG. 18 is a conceptual diagram illustrating an example of the content of a process in which a degree of extension is calculated by the control unit based on a depth measured by a depth sensor (that is, an actually measured depth);

FIG. 19 is a conceptual diagram illustrating examples of a destination to which various kinds of information are to be output; and

FIG. 20 is a conceptual diagram illustrating an example of a series of processing operations in which a processor of the endoscope system provides a process execution request to an external device via a network, the external device executes a process in response to the process execution request, and the processor of the endoscope system receives a process result from the external device.

### DETAILED DESCRIPTION

An example of an embodiment of a medical support device, an endoscope system, a medical support method, and a program according to the technology of the present disclosure will be described hereinafter with reference to the accompanying drawings.

First, terms used in the following description will be described.

CPU is an abbreviation for “Central Processing Unit”. GPU is an abbreviation for “Graphics Processing Unit”. RAM is an abbreviation for “Random Access Memory”. NVM is an abbreviation for “Non-volatile memory”. EEPROM is an abbreviation for “Electrically Erasable Programmable Read-Only Memory”. ASIC is an abbreviation for “Application Specific Integrated Circuit”. PLD is an abbreviation for “Programmable Logic Device”. FPGA is an abbreviation for “Field-Programmable Gate Array”. SoC is an abbreviation for “System-on-a-chip”. SSD is an abbreviation for “Solid State Drive”. USB is an abbreviation for “Universal Serial Bus”. HDD is an abbreviation for “Hard Disk Drive”. EL is an abbreviation for “Electro-Luminescence”. CMOS is an abbreviation for “Complementary Metal Oxide Semiconductor”. CCD is an abbreviation for “Charge Coupled Device”. AI is an abbreviation for “Artificial Intelligence”. BLI is an abbreviation for “Blue Light Imaging”. LCI is an abbreviation for “Linked Color Imaging”. I/F is an abbreviation for “Interface”.

SSL is an abbreviation for “Sessile Serrated Lesion”. LAN is an abbreviation for “Local Area Network”. WAN is an abbreviation for “Wide Area Network”.

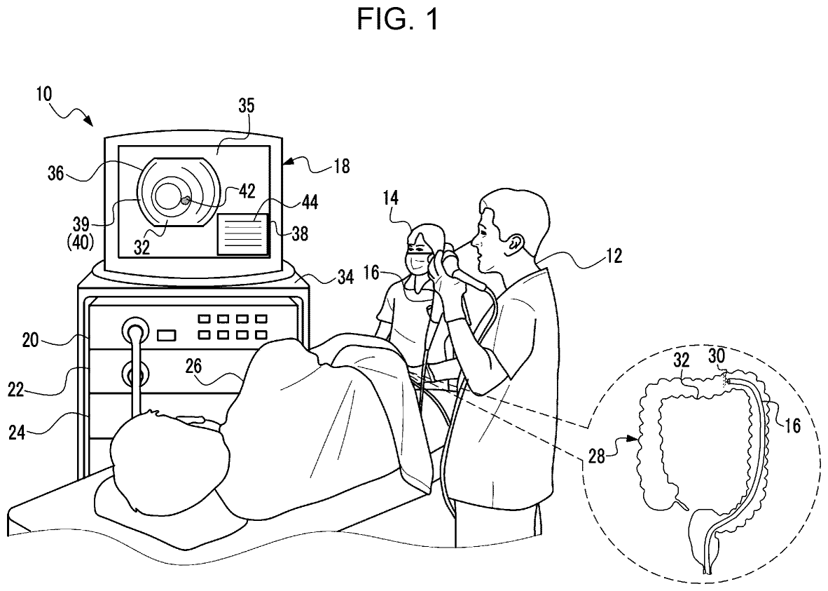

As an example, as illustrated in FIG. 1, an endoscope system 10 is used by a doctor 12 in an endoscopic examination. The endoscopic examination is assisted by staff such as a nurse 14. In the present embodiment, the endoscope system 10 is an example of the “endoscope system” according to the technology of the present disclosure.

The endoscope system 10 is connected to a communication device (not illustrated) in a communicable manner, and information obtained by the endoscope system 10 is transmitted to the communication device. An example of the communication device is a server and/or a client terminal (for example, a personal computer and/or a tablet terminal) that manages various kinds of information such as electronic medical records. The communication device receives the information transmitted from the endoscope system 10 and executes a process using the received information (for example, a process of storing the information in an electronic medical record or the like).

The endoscope system 10 includes an endoscope 16, a display device 18, a light source device 20, a control device 22, and a medical support device 24. In the present embodiment, the endoscope 16 is an example of the “endoscope” according to the technology of the present disclosure.

The endoscope system 10 is an apparatus for performing medical care for a large intestine 28 included in the body of a subject 26 (for example, a patient) using the endoscope 16. In the present embodiment, the large intestine 28 is a target to be observed by the doctor 12.

The endoscope 16 is used by the doctor 12 and is inserted into a body cavity of the subject 26. In the present embodiment, the endoscope 16 is inserted into the large intestine 28 of the subject 26. The endoscope system 10 causes the endoscope 16 inserted into the large intestine 28 of the subject 26 to perform imaging of the inside of the large intestine 28 of the subject 26, and performs various medical treatments on the large intestine 28 as necessary.

The endoscope system 10 performs imaging of the inside of the large intestine 28 of the subject 26 to acquire an image indicating the state of the inside of the large intestine 28, and outputs the acquired image. In the present embodiment, the endoscope system 10 is an endoscope having an optical imaging function of capturing an image of reflected light obtained by irradiating the inside of the large intestine 28 with light 30 and reflecting the light 30 from an intestinal wall 32 of the large intestine 28.

While the endoscopic examination of the large intestine 28 is exemplified here, this is merely an example, and the technology of the present disclosure is also applicable to an endoscopic examination of a luminal organ such as the esophagus, the stomach, the duodenum, or the trachea.

The light source device 20, the control device 22, and the medical support device 24 are installed in a cart 34. The cart 34 is provided with a plurality of shelves along the vertical direction, and the medical support device 24, the control device 22, and the light source device 20 are installed on the shelves from bottom to top. The display device 18 is installed on top of the cart 34.

The control device 22 performs overall control of the endoscope system 10. Under the control of the control device 22, the medical support device 24 performs various kinds of image processing on an image obtained by imaging the intestinal wall 32 with the endoscope 16.

The display device 18 displays various kinds of information including images. Examples of the display device 18 include a liquid crystal display and an EL display. A tablet terminal with a display may be used instead of or together with the display device 18.

The display device 18 displays a screen 35. The screen 35 includes a plurality of display regions. The plurality of display regions are arranged side by side on the screen 35. In the example illustrated in FIG. 1, a first display region 36 and a second display region 38 are depicted as an example of the plurality of display regions. The size of the first display region 36 is larger than the size of the second display region 38. The first display region 36 is used as a main display region, and the second display region 38 is used as a sub-display region. The relationship in size between the first display region 36 and the second display region 38 is not limited to this, and the first display region 36 and the second display region 38 may have any relationship in size so as to fit in the screen 35.

The first display region 36 displays an endoscopic moving image 39. The endoscopic moving image 39 is a moving image acquired by imaging the intestinal wall 32 with the endoscope 16 in the large intestine 28 of the subject 26. In the example illustrated in FIG. 1, a moving image in which the intestinal wall 32 appears is depicted as an example of the endoscopic moving image 39.

The intestinal wall 32 appearing in the endoscopic moving image 39 includes a lesion 42 (for example, in the example illustrated in FIG. 1, one lesion 42) as a region of interest (that is, an observation target region) to be gazed at by the doctor 12, and the doctor 12 can visually recognize the state of the intestinal wall 32 including the lesion 42 through the endoscopic moving image 39. In the present embodiment, the lesion 42 is an example of the “observation target region” and the “lesion” according to the technology of the present disclosure. The intestinal wall 32 including the lesion 42 is an example of the “imaging target region” according to the technology of the present disclosure.

There are various types of lesions 42, and the types of lesions 42 include, for example, neoplastic polyps and non-neoplastic polyps. The types of the neoplastic polyps include, for example, adenomatous polyps (for example, SSL). The types of the non-neoplastic polyps include, for example, hamartoma polyps, hyperplastic polyps, and inflammatory polyps. The types exemplified here are types considered in advance to be possible types of the lesion 42 when an endoscopic examination is performed on the large intestine 28, and the type of lesion differs depending on the organ on which the endoscopic examination is performed.

While the present embodiment provides an example embodiment in which one lesion 42 appears in the endoscopic moving image 39 for convenience of description, the technology of the present disclosure is not limited to this, and the technology of the present disclosure is also applicable in a case where a plurality of lesions 42 appear in the endoscopic moving image 39.

While the present embodiment exemplifies the lesion 42, this is merely an example. The region of interest (that is, the observation target region) to be gazed at by the doctor 12 may be an organ (for example, the duodenal papilla), a marked region, an artificial treatment tool (for example, an artificial clip), a treated region (for example, a region with a trace of removal of a polyp or the like), or the like.

The image displayed in the first display region 36 is one frame 40 included in a moving image configured to include a plurality of frames 40 along a time series. That is, the first display region 36 displays the plurality of frames 40 along a time series at a predetermined frame rate (for example, several tens of frames/second). In the present embodiment, the frame 40 is an example of the “medical image” and the “endoscopic image” according to the technology of the present disclosure.

An example of the moving image to be displayed in the first display region 36 is a live-view moving image. The live-view moving image is merely an example, and a moving image that is temporarily stored in a memory or the like before being displayed, like a post-view moving image, may be used. Alternatively, each frame included in a recording moving image stored in the memory or the like may be reproduced and displayed on the screen 35 (for example, the first display region 36) as the endoscopic moving image 39.

On the screen 35, the second display region 38 is adjacent to the first display region 36 and is displayed in a lower right portion of the screen 35 when viewed from the front. The second display region 38 may be displayed at any position within the screen 35 of the display device 18, but is preferably displayed at a position that enables comparison with the endoscopic moving image 39. The second display region 38 displays auxiliary information 44 for assisting the doctor 12 in making a medical determination or the like. Examples of the auxiliary information 44 include various kinds of information related to the subject 26 into which the endoscope 16 is inserted, and/or various kinds of information obtained by performing processing using AI on the endoscopic moving image 39.

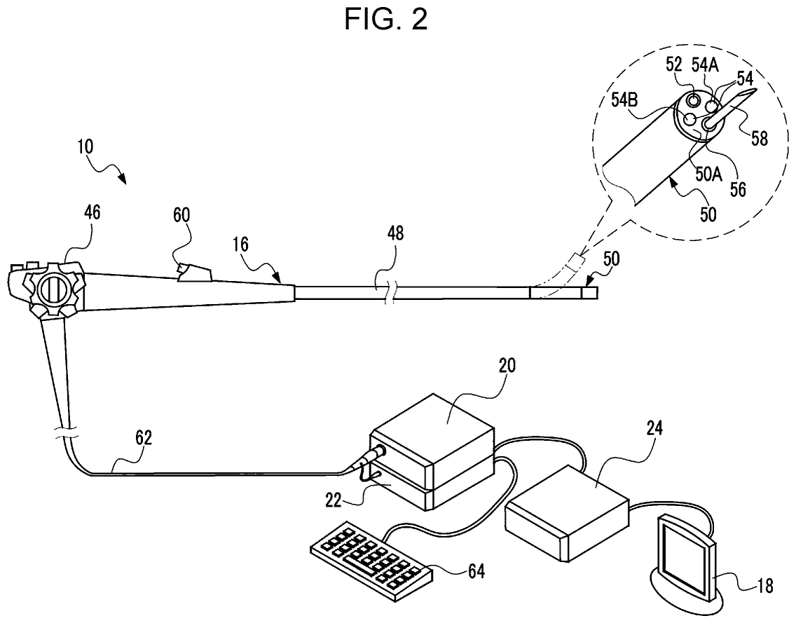

As an example, as illustrated in FIG. 2, the endoscope 16 includes an operation section 46 and an insertion section 48. The insertion section 48 partially bends in response to the operation section 46 being operated. When the doctor 12 (see FIG. 1) operates the operation section 46, the insertion section 48 is inserted into the large intestine 28 (see FIG. 1) while bending according to the shape of the large intestine 28.

The insertion section 48 has a tip portion 50 provided with a camera 52, an illumination device 54, and a treatment tool opening 56. The camera 52 and the illumination device 54 are provided on a tip surface 50A of the tip portion 50. While an example embodiment in which the camera 52 and the illumination device 54 are provided on the tip surface 50A of the tip portion 50 is given here, this is merely an example. The camera 52 and the illumination device 54 may be provided on a side surface of the tip portion 50 such that the endoscope 16 is configured as a side view endoscope.

The camera 52 is inserted into the body cavity of the subject 26 to perform imaging of an observation target region. In the present embodiment, the camera 52 performs imaging of the inside of the body of the subject 26 (for example, the inside of the large intestine 28) to acquire the endoscopic moving image 39. An example of the camera 52 is a CMOS camera. However, this is merely an example, and the camera 52 may be any other type of camera such as a CCD camera. The camera 52 is an example of the “camera” according to the technology of the present disclosure.

The illumination device 54 has illumination windows 54A and 54B. The illumination device 54 emits the light 30 (see FIG. 1) through the illumination windows 54A and 54B. Examples of the type of the light 30 to be emitted from the illumination device 54 include visible light (for example, white light or the like) and invisible light (for example, near-infrared light or the like). The illumination device 54 further emits special light through the illumination windows 54A and 54B. Examples of the special light include light for BLI and/or light for LCI. The camera 52 performs imaging of the inside of the large intestine 28 using an optical method, with the inside of the large intestine 28 irradiated with the light 30 from the illumination device 54.

The treatment tool opening 56 is an opening for allowing a treatment tool 58 to protrude from the tip portion 50. The treatment tool opening 56 is also used as a suction port for sucking blood, bodily waste, and the like, and as a delivery port for delivering a fluid.

The operation section 46 has a treatment tool insertion port 60 formed therein, and the treatment tool 58 is inserted into the insertion section 48 through the treatment tool insertion port 60. The treatment tool 58 passes through the insertion section 48 and protrudes to the outside from the treatment tool opening 56. In the example illustrated in FIG. 2, a puncture needle is illustrated as the treatment tool 58 protruding from the treatment tool opening 56. While a puncture needle is exemplified as the treatment tool 58, this is merely an example, and the treatment tool 58 may be gripping forceps, a papillotomy knife, a snare, a catheter, a guide wire, a cannula, a puncture needle with a guide sheath, and/or the like.

The endoscope 16 is connected to the light source device 20 and the control device 22 through a universal cord 62. The control device 22 is connected to the medical support device 24 and a reception device 64. The medical support device 24 is also connected to the display device 18. That is, the control device 22 is connected to the display device 18 through the medical support device 24.

Since the medical support device 24 is exemplified here as an external device for extending the functions implemented by the control device 22, an example embodiment in which the control device 22 and the display device 18 are indirectly connected through the medical support device 24 is given here, although this is merely an example. For example, the display device 18 may be directly connected to the control device 22. In this case, for example, the control device 22 may be mounted with the functions of the medical support device 24, or the control device 22 may be mounted with a function of causing a server (not illustrated) to execute the same process as a process (for example, a medical support process described below) executed by the medical support device 24 and receiving and using a process result obtained by the server.

The reception device 64 receives an instruction from the doctor 12 and outputs the received instruction to the control device 22 as an electrical signal. An example of the reception device 64 is a keyboard, a mouse, a touch panel, a foot switch, a microphone, and/or a remote operation device.

The control device 22 controls the light source device 20, transmits and receives various signals to and from the camera 52, and transmits and receives various signals to and from the medical support device 24.

The light source device 20 emits light under the control of the control device 22 and supplies the light to the illumination device 54. The illumination device 54 incorporates a light guide, and the light supplied from the light source device 20 is emitted from the illumination windows 54A and 54B through the light guide. The control device 22 causes the camera 52 to perform imaging, acquires the endoscopic moving image 39 (see FIG. 1) from the camera 52, and outputs the endoscopic moving image 39 to a predetermined output destination (for example, the medical support device 24).

The medical support device 24 performs various kinds of image processing on the endoscopic moving image 39 input from the control device 22 to support medical treatment (here, as an example, endoscopy). The medical support device 24 outputs the endoscopic moving image 39 on which the various kinds of image processing have been performed to a predetermined output destination (for example, the display device 18).

While an example embodiment has been described in which the endoscopic moving image 39 output from the control device 22 is output to the display device 18 through the medical support device 24, this is merely an example. For example, in another aspect, the control device 22 and the display device 18 may be connected to each other, and the endoscopic moving image 39 on which image processing has been performed by the medical support device 24 may be displayed on the display device 18 through the control device 22.

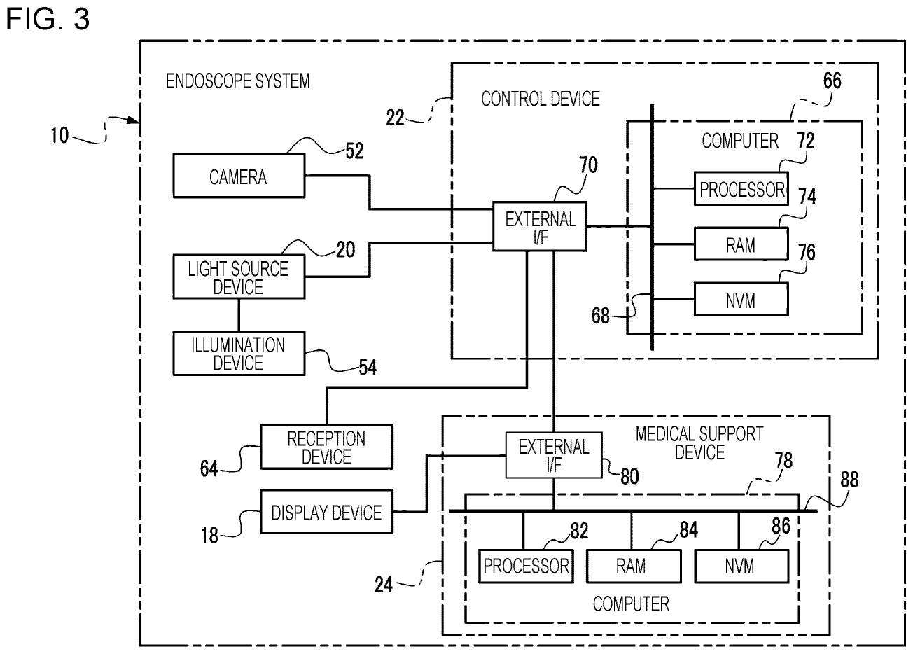

As an example, as illustrated in FIG. 3, the control device 22 includes a computer 66, a bus 68, and an external I/F 70. The computer 66 includes a processor 72, a RAM 74, and an NVM 76. The processor 72, the RAM 74, the NVM 76, and the external I/F 70 are connected to the bus 68.

For example, the processor 72 has at least one CPU and at least one GPU and controls the entire control device 22. The GPU operates under the control of the CPU and is responsible for performing various kinds of graphics-based processing, arithmetic operations using a neural network, and the like. The processor 72 may include one or more CPUs with integrated GPU functions, or may include one or more CPUs without integrated GPU functions. In the example illustrated in FIG. 3, the computer 66 is mounted with one processor 72. However, this is merely an example, and the computer 66 may be mounted with a plurality of processors 72.

The RAM 74 is a memory that temporarily stores information and is used as a work memory by the processor 72. The NVM 76 is a non-volatile storage device that stores various programs, various parameters, and the like. An example of the NVM 76 is a flash memory (for example, an EEPROM and/or an SSD). The flash memory is merely an example, and the NVM 76 may be any other non-volatile storage device such as an HDD, or a combination of two or more types of non-volatile storage devices.

The external I/F 70 handles transmission and reception of various kinds of information between the processor 72 and one or more devices (hereinafter also referred to as “first external devices”) external to the control device 22. An example of the external I/F 70 is a USB interface.

The camera 52 is connected to the external I/F 70 as one of the first external devices, and the external I/F 70 handles transmission and reception of various kinds of information between the camera 52 and the processor 72. The processor 72 controls the camera 52 through the external I/F 70. Further, the processor 72 acquires the endoscopic moving image 39 (see FIG. 1), which is obtained by imaging the inside of the large intestine 28 (see FIG. 1) using the camera 52, through the external I/F 70.

The light source device 20 is connected to the external I/F 70 as one of the first external devices, and the external I/F 70 handles transmission and reception of various kinds of information between the light source device 20 and the processor 72. The light source device 20 supplies light to the illumination device 54 under the control of the processor 72. The illumination device 54 emits the light supplied from the light source device 20.

The reception device 64 is connected to the external I/F 70 as one of the first external devices, and the processor 72 acquires an instruction received by the reception device 64 through the external I/F 70 and executes a process corresponding to the acquired instruction.

The medical support device 24 includes a computer 78 and an external I/F 80. The computer 78 includes a processor 82, a RAM 84, and an NVM 86. The processor 82, the RAM 84, the NVM 86, and the external I/F 80 are connected to a bus 88. In the present embodiment, the medical support device 24 is an example of the “medical support device” according to the technology of the present disclosure, the computer 78 is an example of the “computer” according to the technology of the present disclosure, and the processor 82 is an example of the “processor” according to the technology of the present disclosure.

Since the hardware configuration (that is, the processor 82, the RAM 84, and the NVM 86) of the computer 78 is basically the same as the hardware configuration of the computer 66, the description of the hardware configuration of the computer 78 will be omitted here.

The external I/F 80 handles transmission and reception of various kinds of information between the processor 82 and one or more devices (hereinafter also referred to as “second external devices”) external to the medical support device 24. An example of the external I/F 80 is a USB interface.

The control device 22 is connected to the external I/F 80 as one of the second external devices. In the example illustrated in FIG. 3, the external I/F 70 of the control device 22 is connected to the external I/F 80. The external I/F 80 handles transmission and reception of various kinds of information between the processor 82 of the medical support device 24 and the processor 72 of the control device 22. For example, the processor 82 acquires the endoscopic moving image 39 (see FIG. 1) from the processor 72 of the control device 22 through the external I/Fs 70 and 80, and performs various kinds of image processing on the acquired endoscopic moving image 39.

The display device 18 is connected to the external I/F 80 as one of the second external devices. The processor 82 controls the display device 18 through the external I/F 80 to display various kinds of information (for example, the endoscopic moving image 39 and the like on which the various kinds of image processing have been performed) on the display device 18.

In an endoscopic examination, the doctor 12 determines whether the lesion 42 appearing in the endoscopic moving image 39 requires medical treatment, while checking the endoscopic moving image 39 through the display device 18, and performs medical treatment on the lesion 42, if necessary. The size of the lesion 42 is a determination factor important for determining whether medical treatment is necessary.

The recent development of machine learning has enabled the AI-based detection and classification of the lesion 42 based on the endoscopic moving image 39. Application of this technique makes it possible to measure the size of the lesion 42 from the endoscopic moving image 39. Accurately measuring the size of the lesion 42 and presenting the measurement result to the doctor 12 is very useful for the doctor 12 to perform medical treatment on the lesion 42.

However, depending on the appearance of the lesion 42 in the endoscopic moving image 39 (for example, the appearance of the lesion 42 in the endoscopic moving image 39 in a case where the relative positional relationship between the lesion 42 and the camera 52 is not a positional relationship expected in advance), the size of the lesion 42 may be erroneously measured.

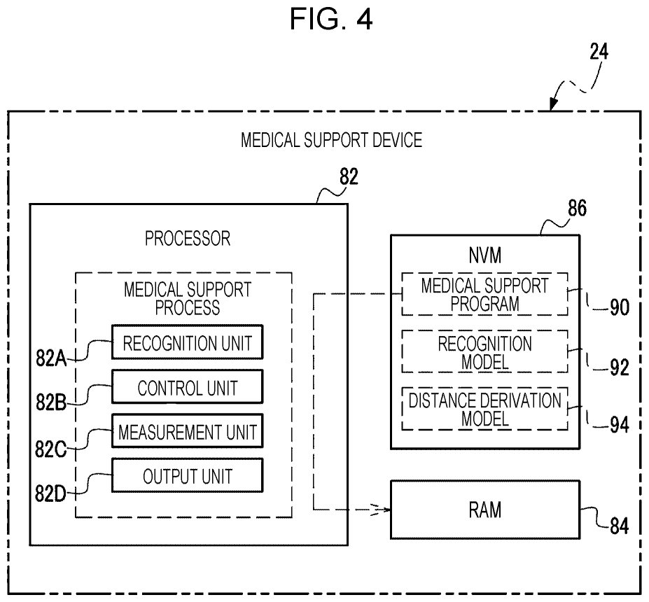

In view of such circumstances, in the present embodiment, as an example, as illustrated in FIG. 4, the processor 82 of the medical support device 24 performs a medical support process.

The NVM 86 stores a medical support program 90. The medical support program 90 is an example of the “program” according to the technology of the present disclosure. The processor 82 reads the medical support program 90 from the NVM 86 and executes the read medical support program 90 on the RAM 84 to perform the medical support process. The medical support process is implemented by the processor 82 operating as a recognition unit 82A, a control unit 82B, a measurement unit 82C, and an output unit 82D in accordance with the medical support program 90 executed on the RAM 84.

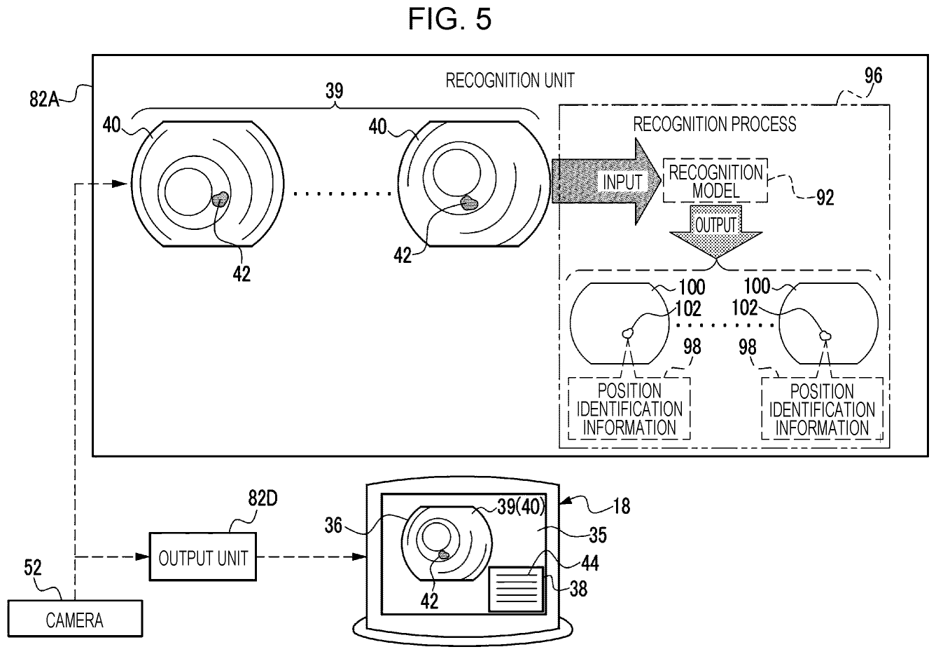

The NVM 86 stores a recognition model 92 and a distance derivation model 94. As described in detail below, the recognition model 92 is used by the recognition unit 82A, and the distance derivation model 94 is used by the control unit 82B and the measurement unit 82C. As an example, as illustrated in FIG. 5, the recognition unit 82A and the output unit 82D acquire each of the plurality of frames 40 along a time series included in the endoscopic moving image 39 generated by imaging with the camera 52 in accordance with an imaging frame rate (for example, several tens of frames/second) from the camera 52 frame by frame along a time series.

The output unit 82D outputs the endoscopic moving image 39 to the display device 18. For example, the output unit 82D displays the endoscopic moving image 39 in the first display region 36 as a live view image. That is, each time the output unit 82D acquires a frame 40 from the camera 52, the output unit 82D sequentially displays the acquired frame 40 in the first display region 36 in accordance with a display frame rate (for example, several tens of frames/second). The output unit 82D further displays the auxiliary information 44 in the second display region 38. Further, for example, the output unit 82D updates the content (for example, the auxiliary information 44) displayed in the second display region 38 in accordance with the content displayed in the first display region 36.

The recognition unit 82A uses the endoscopic moving image 39 acquired from the camera 52 to recognize the lesion 42 in the endoscopic moving image 39. That is, the recognition unit 82A sequentially performs a recognition process 96 on each of the plurality of frames 40 along a time series included in the endoscopic moving image 39 acquired from the camera 52 to recognize the lesion 42 appearing in each of the frames 40. For example, the recognition unit 82A recognizes the geometric characteristics (for example, the position, the shape, and the like) of the lesion 42, the type of the lesion 42, the category of the lesion 42 (for example, pedunculated, sub-pedunculated, sessile, superficial elevated, superficial flat, superficial depressed, and the like), and the like.

Each time a frame 40 is acquired, the recognition unit 82A performs the recognition process 96 on the acquired frame 40. The recognition process 96 is a process for recognizing the lesion 42 by a method using AI. In the present embodiment, for example, an object recognition process using an AI-based segmentation method (for example, semantic segmentation, instance segmentation, and/or panoptic segmentation) is used as the recognition process 96.

A process using the recognition model 92 is performed as the recognition process 96. The recognition model 92 is a trained model for object recognition using an AI-based segmentation method. An example of the trained model for object recognition using an AI-based segmentation method is a model for semantic segmentation. An example of the model for semantic segmentation is an encoder-decoder structure model. An example of the encoder-decoder structure model is a U-Net model, an HRNet model, or the like. In the present embodiment, the recognition process 96 is an example of the “object recognition process” according to the technology of the present disclosure.

The recognition model 92 is optimized by training a neural network through machine learning using first training data. The first training data is a dataset including a plurality of pieces of data (that is, data for a plurality of frames) in which first example data and first ground-truth data are associated with each other.

The first example data is an image corresponding to the frame 40. The first ground-truth data is ground-truth data (that is, an annotation) for the first example data. An example of the first ground-truth data is an annotation for identifying the geometric characteristics, type, and category of a lesion appearing in an image used as the first example data.

The recognition unit 82A acquires a frame 40 from the camera 52 and inputs the acquired frame 40 to the recognition model 92. Accordingly, each time a frame 40 is input, the recognition model 92 identifies the geometric characteristics of the lesion 42 appearing in the input frame 40 and outputs information that can identify the geometric characteristics. In the example illustrated in FIG. 5, position identification information 98 that can identify the position of the lesion 42 in the frame 40 is depicted as an example of the information that can identify the geometric characteristics. Further, the recognition unit 82A acquires, from the recognition model 92, information indicating the type and category of the lesion 42 appearing in the frame 40 input to the recognition model 92.

Each time a frame 40 is input to the recognition model 92, the recognition unit 82A acquires, from the recognition model 92, a probability map 100 related to the frame 40 input to the recognition model 92. The probability map 100 is a map in which the distribution of the position of the lesion 42 in the frame 40 is expressed in terms of a probability, which is an example of a measure of the likelihood. The probability map 100 is typically referred to also as a reliability map, a certainty map, or the like.

The probability map 100 includes a segmentation image 102 that defines the lesion 42 recognized by the recognition unit 82A. The segmentation image 102 is an image region for identifying the position of the lesion 42, which is recognized by performing the recognition process 96 on the frame 40, in the frame 40 (that is, an image displayed in a display style that can identify the position where the lesion 42 is most likely to be present in the frame 40). The segmentation image 102 is associated with the position identification information 98 by the recognition unit 82A. Examples of the position identification information 98 in this case include coordinates for identifying the position of the segmentation image 102 in the frame 40. The probability map 100 may be displayed on the screen 35 (for example, the second display region 38) as the auxiliary information 44 by the output unit 82D. In this case, the probability map 100 displayed on the screen 35 is updated in accordance with the display frame rate applied to the first display region 36. That is, the display of the probability map 100 in the second display region 38 (that is, the display of the segmentation image 102) is updated in synchronization with the display timing of the endoscopic moving image 39 displayed in the first display region 36. This configuration allows the doctor 12 to grasp the schematic position of the lesion 42 in the endoscopic moving image 39 displayed in the first display region 36 by referring to the probability map 100 displayed in the second display region 38 while observing the endoscopic moving image 39 displayed in the first display region 36.

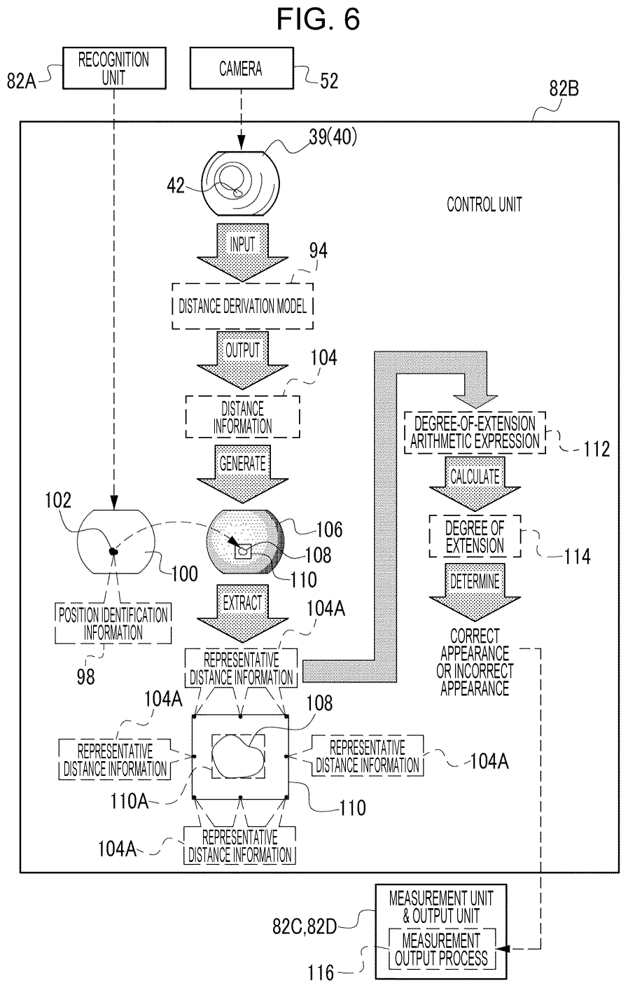

As an example, as illustrated in FIG. 6, the control unit 82B acquires a frame 40 included in the endoscopic moving image 39 (as an example, the frame 40 used in the recognition process 96) from the camera 52, and acquires, from the recognition unit 82A, a recognition result obtained by performing the recognition process 96 (see FIG. 5) on the frame 40. The control unit 82B determines whether the appearance of the lesion 42 in the frame 40 is correct, based on the frame 40 acquired from the camera 52 and the recognition result acquired from the recognition unit 82A.

The correct appearance refers to, for example, an appearance in which the relative positional relationship between the lesion 42 and the camera 52 is a positional relationship expected in advance. Examples of the positional relationship expected in advance include a positional relationship in which the camera 52 directly faces a lesion surface region. The lesion surface region refers to a surface region that is a portion of the imaging target region (that is, the entire region, in real space, appearing in the frame 40) and that includes the lesion 42. Examples of the positional relationship in which the camera 52 directly faces the lesion surface region include a positional relationship in which the optical axis of the camera 52 is perpendicular to the lesion surface region within an allowable error. The allowable error refers to an error allowed in advance as an error by which the correct size of the lesion 42 in real space can be measured when the size of the lesion 42 is measured by the measurement unit 82C.

To implement the determination of whether the appearance of the lesion 42 in the frame 40 is correct, the control unit 82B acquires distance information 104 of the lesion 42, based on the frame 40 acquired from the camera 52. The distance information 104 is information indicating the distance from the camera 52 (that is, the observation position) to the intestinal wall 32 (see FIG. 1) including the lesion 42.

While the distance from the camera 52 to the intestinal wall 32 including the lesion 42 is exemplified here, this is merely an example. Instead of the distance, a numerical value indicating the depth from the camera 52 to the intestinal wall 32 including the lesion 42 (for example, a plurality of numerical values defining depths in a stepwise manner (for example, numerical values in several steps to several tens of steps)) may be used.

The distance information 104 is acquired for each of all the pixels constituting the frame 40. The distance information 104 may be acquired for each block (for example, a pixel group constituted by several pixels to several hundreds of pixels), which is larger than a pixel in the frame 40.

The control unit 82B acquires the distance information 104 by, for example, deriving the distance information 104 by using an AI-based method. In the present embodiment, the distance derivation model 94 is used to derive the distance information 104.

The distance derivation model 94 is optimized by training the neural network through machine learning using second training data. The second training data is a dataset including a plurality of pieces of data (that is, data for a plurality of frames) in which second example data and second ground-truth data are associated with each other.

The second example data is an image corresponding to the frame 40. The second ground-truth data is ground-truth data (that is, an annotation) for the second example data. An example of the second ground-truth data is an annotation for identifying a distance corresponding to each pixel appearing in an image used as the second example data.

The control unit 82B acquires the frame 40 from the camera 52 and inputs the acquired frame 40 to the distance derivation model 94. As a result, the distance derivation model 94 outputs the distance information 104 on a pixel-by-pixel basis in the frame 40 that has been input. That is, in the control unit 82B, information indicating the distance from the position of the camera 52 (for example, the position of the image sensor, the objective lens, or the like mounted in the camera 52) to the intestinal wall 32 appearing in the frame 40 is output from the distance derivation model 94 as the distance information 104 on a pixel-by-pixel basis in the frame 40.

The control unit 82B generates a distance image 106, based on the distance information 104 output from the distance derivation model 94. The distance image 106 is an image in which the distance information 104 is distributed in units of pixels included in the endoscopic moving image 39.

The control unit 82B acquires the position identification information 98 assigned to the segmentation image 102 on the probability map 100 obtained by the recognition unit 82A. The control unit 82B extracts representative distance information 104A from a partial distance image 110 determined based on a segmentation-corresponding region 108 in the distance image 106.

The segmentation-corresponding region 108 is a closed region corresponding to the position of the segmentation image 102 in the distance image 106. The partial distance image 110 is determined based on the segmentation-corresponding region 108. For example, the partial distance image 110 is a distance image cropped from the distance image 106 using a rectangular frame obtained by enlarging a rectangular frame 110A circumscribing the segmentation-corresponding region 108 by a predetermined magnification. Examples of the predetermined magnification include a magnification exceeding “1” (for example, a magnification of 2). The predetermined magnification may be a fixed value or a variable value that is changed in accordance with an instruction received by the reception device 64 and/or various conditions. While the rectangular frame 110A circumscribing the segmentation-corresponding region 108 is exemplified here, this is merely an example. A geometric shape frame (for example, a circle or the like) circumscribing the segmentation-corresponding region 108 or a frame obtained by enlarging the outer contour of the segmentation-corresponding region 108 by a predetermined magnification may be used.

The representative distance information 104A is distance information 104 obtained from each of a plurality of locations in the partial distance image 110. The plurality of locations are at least two locations including one location and the other location between which the segmentation-corresponding region 108 is crossed. Examples of crossing the segmentation-corresponding region 108 include crossing the segmentation-corresponding region 108 by the longest distance and crossing the segmentation-corresponding region 108 by the shortest distance.

In the example illustrated in FIG. 6, distance information 104 obtained from each of eight locations in the partial distance image 110 is illustrated as the representative distance information 104A. The eight locations in the partial distance image 110 are the four vertices and the midpoint of each side of the partial distance image 110. This is merely an example, and it is sufficient to obtain, as a plurality of pieces of representative distance information 104A (here, as an example, eight pieces of representative distance information 104A), pieces of distance information 104 for a plurality of locations (here, as an example, eight locations) that can identify the extent to which the lesion 42 extends to the side deeper into the body cavity (that is, in the depth direction of the large intestine 28) when the lesion 42 is viewed from the camera 52 (in other words, the degree to which the camera 52 directly faces the lesion 42).

The control unit 82B calculates a degree of extension 114, which is an example of information related to an extension of the lesion surface region from the camera 52 side in the depth direction, by using a degree-of-extension arithmetic expression 112. The degree of extension 114 refers to the degree to which the lesion surface region extends from the camera 52 side in the depth direction. For example, the degree of extension 114 is a measure that can be determined from a difference between a maximum value and a minimum value of the plurality of pieces of representative distance information 104A, a standard deviation, and/or the like.

The degree-of-extension arithmetic expression 112 is an arithmetic expression in which the plurality of pieces of representative distance information 104A are dependent variables and the degree of extension 114 is an independent variable. The control unit 82B inputs the plurality of pieces of representative distance information 104A extracted from the partial distance image 110 to the degree-of-extension arithmetic expression 112. The degree-of-extension arithmetic expression 112 outputs a degree of extension 114 corresponding to the plurality of pieces of representative distance information 104A that have been input.

In the present embodiment, the degree of extension 114 is defined by two levels, “high” and “low”. The “high” level indicates that the degree to which the lesion surface region extends from the camera 52 side in the depth direction is large, and the “low” level indicates that the degree to which the lesion surface region extends from the camera 52 side in the depth direction is small. Here, a large degree of extension is synonymous with a meaning that the degree of extension is so large that the correct size of the lesion 42 cannot be measured when the measurement unit 82C measures the size of the lesion 42, and a small degree of extension is synonymous with a meaning that the degree of extension is small enough that the correct size of the lesion 42 can be measured when the measurement unit 82C measures the size of the lesion 42.

The control unit 82B determines whether the appearance of the lesion 42 in the frame 40 is correct, based on the degree of extension 114. For example, when the degree of extension 114 is “high”, the control unit 82B determines that the appearance of the lesion 42 in the frame 40 is incorrect, and when the degree of extension 114 is “low”, the control unit 82B determines that the appearance of the lesion 42 in the frame 40 is correct. In the present embodiment, the degree of extension is represented in two levels to facilitate understanding of the technology of the present disclosure. However, this is merely an example, and the degree of extension may be information (for example, a numerical value) evaluated in three or more levels or may be information (for example, a numerical value) represented in a continuous manner.

The control unit 82B controls a measurement output process 116 in accordance with the degree of extension 114. The measurement output process 116 is a process of measuring the size of the lesion 42 based on the frame 40 and outputting the size of the lesion 42, and is executed by the measurement unit 82C and the output unit 82D. In the present embodiment, the control unit 82B causes the measurement unit 82C and the output unit 82D to execute the measurement output process 116 when the control unit 82B determines that the appearance of the lesion 42 in the frame 40 is correct.

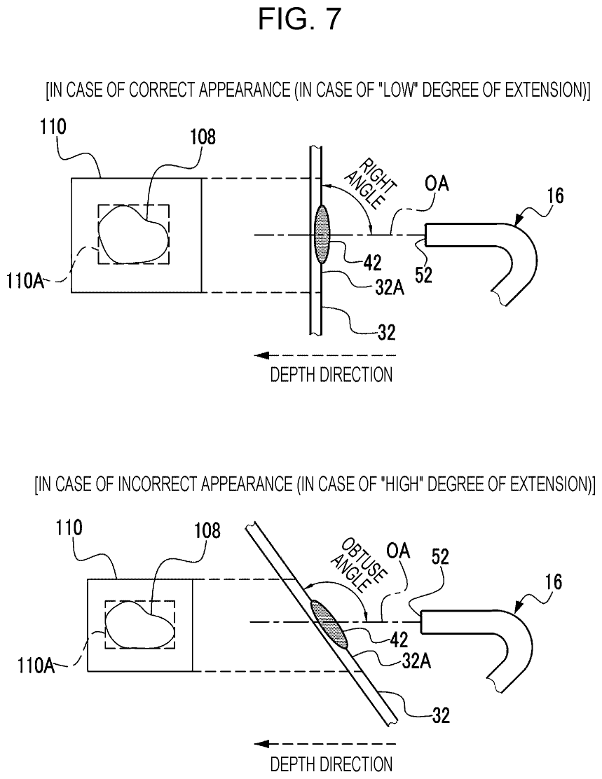

In an example illustrated in FIG. 7, a surface region 32A is depicted as an example of the lesion surface region described above. The surface region 32A is a surface region present at a position identified from the partial distance image 110 within the intestinal wall 32, which is in the angle of view of the camera 52. When the relative positional relationship between the surface region 32A and the camera 52 is a positional relationship in which the surface region 32A and the camera 52 directly face each other, that is, when the degree of extension 114 is “low”, the control unit 82B determines that the appearance of the lesion 42 in the frame 40 is correct. In this case, the measurement output process 116 is executed by the measurement unit 82C and the output unit 82D (see FIG. 6). In the present embodiment, the positional relationship in which the surface region 32A and the camera 52 directly face each other and the “low” degree of extension 114 are examples of the “predetermined positional relationship” according to the technology of the present disclosure.

By contrast, when the relative positional relationship between the surface region 32A and the camera 52 is a positional relationship in which the surface region 32A and the camera 52 do not directly face each other, that is, when the degree of extension 114 is “high”, the control unit 82B determines that the appearance of the lesion 42 in the frame 40 is incorrect. In this case, the measurement output process 116 is not executed by the measurement unit 82C and the output unit 82D. This is because, even if the size of the lesion 42 in the frame 40 obtained by imaging the intestinal wall 32 including the lesion 42 with the camera 52 is measured by the measurement unit 82C when the degree of extension 114 is “high”, the measurement may be erroneous. In the example illustrated in FIG. 7, when the lesion 42 is viewed from the camera 52 side, the surface region 32A extends in the depth direction. Thus, even if the lesion 42 is recognized by the recognition unit 82A based on the frame 40 obtained by imaging with the camera 52 in this state and the size of the lesion 42 is measured by the measurement unit 82C, the size is measured to be smaller than the actual size of the lesion 42.

In the present embodiment, accordingly, the execution of the measurement output process 116 is controlled in accordance with appearance information related to the appearance of the surface region 32A in the frame 40 (hereinafter simply referred to also as “appearance information”). In the present embodiment, as an example, positional relationship identification information that can identify the relative positional relationship between the camera 52 and the surface region 32A (hereinafter simply referred to also as “positional relationship identification information”) is used as the appearance information. In the present embodiment, as an example, the degree of extension 114 is used as the positional relationship identification information. For this reason, as described above, the measurement output process 116 is executed in accordance with the degree of extension 114. That is, the measurement output process 116 is executed when the degree of extension 114 is “low”, and the measurement output process 116 is not executed when the degree of extension 114 is “high”. In the present embodiment, the degree of extension 114 is an example of the “extension information” according to the technology of the present disclosure.

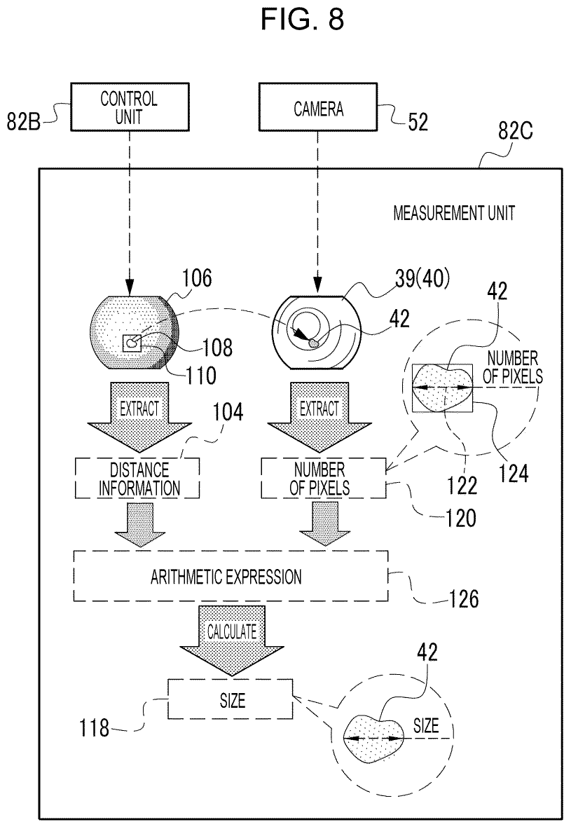

As an example, as illustrated in FIG. 8, in the measurement output process 116, the measurement unit 82C measures a size 118 of the lesion 42. The size 118 of the lesion 42 refers to the size of the lesion 42 in real space.

To implement the measurement of the size 118 of the lesion 42, the measurement unit 82C acquires, from the control unit 82B, the distance image 106 used in the determination performed by the control unit 82B. Then, the measurement unit 82C extracts the distance information 104 from the partial distance image 110 included in the distance image 106. The distance information 104 extracted from the partial distance image 110 includes, for example, distance information 104 corresponding to the position (for example, centroid) of the lesion 42, or the statistical value (for example, median value, mean value, or mode value) of pieces of distance information 104 for a plurality of pixels (for example, all the pixels) included in the lesion 42.

The measurement unit 82C extracts the number of pixels 120 from the frame 40 acquired from the camera 52 (for example, the frame 40 input to the distance derivation model 94 by the control unit 82B). The number of pixels 120 is the number of pixels on a line segment 122 crossing an image region in the frame 40 corresponding to the partial distance image 110, that is, an image region at a position identified from the position identification information 98 within the entire image region of the frame 40 (in other words, an image region indicating the lesion 42). An example of the line segment 122 is the longest line segment parallel to the long sides of a rectangular frame 124 circumscribing the image region indicating the lesion 42. The line segment 122 is merely an example. Instead of the line segment 122, the longest line segment parallel to the short sides of the rectangular frame 124 circumscribing the image region indicating the lesion 42 may be used.

The measurement unit 82C calculates the size 118 of the lesion 42, based on the distance information 104 extracted from the distance image 106 and the number of pixels 120 extracted from the frame 40. The size 118 is calculated using an arithmetic expression 126. The measurement unit 82C inputs the distance information 104 extracted from the distance image 106 and the number of pixels 120 extracted from the frame 40 to the arithmetic expression 126. The arithmetic expression 126 is an arithmetic expression in which the distance information 104 and the number of pixels 120 are independent variables and the size 118 is a dependent variable. The arithmetic expression 126 outputs the size 118 corresponding to the distance information 104 and the number of pixels 120 that have been input.

While the length of the lesion 42 in real space is exemplified as the size 118, the technology of the present disclosure is not limited to this. The size 118 may be the surface area or volume of the lesion 42 in real space. In this case, for example, as the arithmetic expression 126, an arithmetic expression is used in which the number of pixels in the entire image region indicating the lesion 42 and the distance information 104 are independent variables and the surface area or volume of the lesion 42 in real space is a dependent variable.

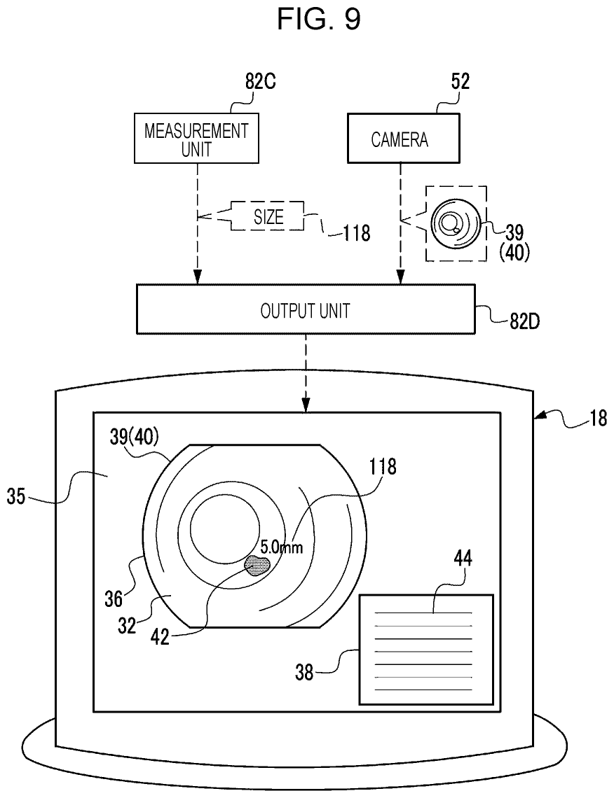

As an example, as illustrated in FIG. 9, in the measurement output process 116, the size 118 measured by the measurement unit 82C is output by displaying the size 118 on the screen 35. In the example illustrated in FIG. 9, the output unit 82D outputs the size 118 measured by the measurement unit 82C to the display device 18, thereby displaying the size 118 on the screen 35. In the example illustrated in FIG. 9, furthermore, the frame 40 is displayed in the first display region 36, and the size 118 is displayed superimposed on the frame 40. The size 118 is displayed at a position near the position of the lesion 42 of which the size 118 is measured.

Each time the measurement unit 82C measures the size 118, the output unit 82D displays the latest size 118 in the first display region 36. That is, the size 118 displayed in the first display region 36 is updated to the latest size 118 each time the size 118 is measured by the measurement unit 82C.

The size 118 may be displayed in the second display region 38 as the auxiliary information 44. The size 118 is preferably displayed on the screen 35 in a display style that can identify the correspondence relationship with the lesion 42 of which the size 118 is measured. In this case, for example, the size 118 and the lesion 42 of which the size 118 is measured may be displayed on the screen 35 in such a manner as to be associated with each other by a line or the like, the size 118 may be displayed in a pop-up manner from the lesion 42 of which the size 118 is measured, a region where the lesion 42 corresponding to the displayed size 118 appears may be displayed in the frame 40 in a more highlighted manner than other regions, or the segmentation image 102 corresponding to the lesion 42 of which the size 118 is measured may be displayed on the probability map 100 in an identifiable display style.

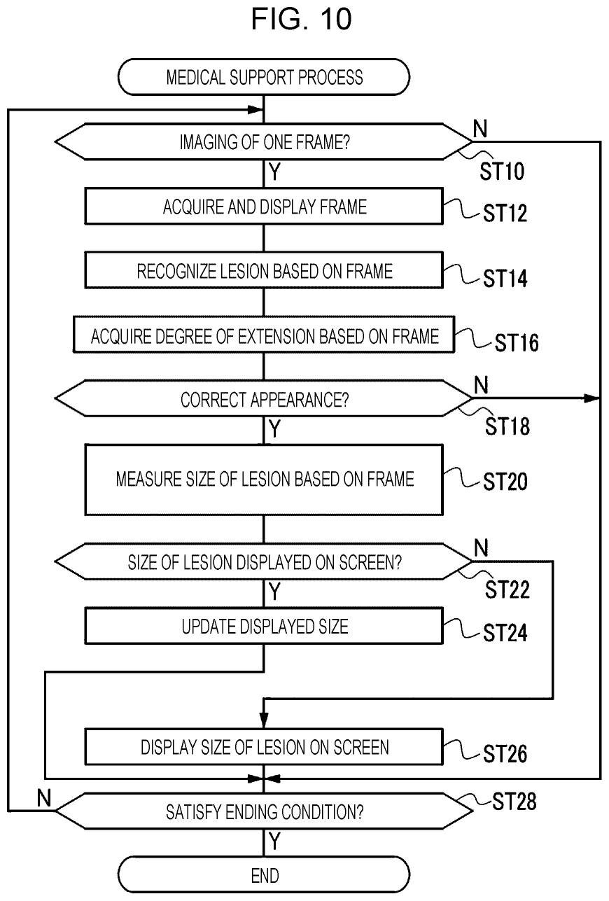

Next, the operation of a portion, according to the technology of the present disclosure, of the endoscope system 10 will be described with reference to FIG. 10. The flow of the medical support process illustrated in FIG. 10 is an example of the “medical support method” according to the technology of the present disclosure.

In the medical support process illustrated in FIG. 10, first, in step ST10, the recognition unit 82A determines whether imaging of one frame has been performed in the large intestine 28 by the camera 52. If imaging of one frame has not been performed in the large intestine 28 by the camera 52 in step ST10, the determination is negative, and the medical support process proceeds to step ST28. If imaging of one frame has been performed in the large intestine 28 by the camera 52 in step ST10, the determination is affirmative, and the medical support process proceeds to step ST12.

In step ST12, the recognition unit 82A and the output unit 82D acquire a frame 40 obtained by imaging the large intestine 28 with the camera 52. Then, the output unit 82D displays the frame 40 in the first display region 36 (see FIGS. 5 and 9). For convenience of description, it is assumed here that the lesion 42 appears in the frame 40. After the processing of step ST12 is performed, the medical support process proceeds to step ST14.

In step ST14, the recognition unit 82A performs the recognition process 96 using the frame 40 acquired in step ST12 to recognize the lesion 42 appearing in the frame 40 (see FIG. 5). After the processing of step ST14 is performed, the medical support process proceeds to step ST16.

In step ST16, the control unit 82B acquires the degree of extension 114, based on the frame 40 acquired in step ST12 and a recognition result obtained by the recognition process 96 performed in step ST14 (see FIG. 6). After the processing of step ST16 is performed, the medical support process proceeds to step ST18.

In step ST18, the control unit 82B determines whether the appearance of the lesion 42 in the frame 40 acquired in step ST12 is correct, based on the degree of extension 114 acquired in step ST16 (see FIG. 6). If the degree of extension 114 acquired in step ST16 is “low”, it is determined that the appearance of the lesion 42 in the frame 40 is correct. If the degree of extension 114 acquired in step ST16 is “high”, it is determined that the appearance of the lesion 42 in the frame 40 is incorrect.

If the appearance of the lesion 42 in the frame 40 is correct in step ST18, the determination is affirmative, and the medical support process proceeds to step ST20. If the appearance of the lesion 42 in the frame 40 is incorrect in step ST18, the determination is negative, and the medical support process proceeds to step ST28.

In step ST20, the measurement unit 82C measures the size 118 of the lesion 42 appearing in the frame 40 acquired in step ST12, based on the frame 40 acquired in step ST12 and the recognition result obtained by the recognition process 96 performed in step ST14 (see FIG. 8). After the processing of step ST20 is performed, the medical support process proceeds to step ST22.

In step ST22, the output unit 82D determines whether the size 118 of the lesion 42 has been displayed in the first display region 36 of the screen 35. If the size 118 of the lesion 42 has been displayed in the first display region 36 of the screen 35 in step ST22, the determination is affirmative, and the medical support process proceeds to step ST24. If the size 118 of the lesion 42 has not been displayed in the first display region 36 of the screen 35 in step ST22, the determination is negative, and the medical support process proceeds to step ST26.

In step ST24, the output unit 82D updates the size 118 displayed in the first display region 36 to the size 118 measured in step ST20 (see FIG. 9). After the processing of step ST24 is performed, the medical support process proceeds to step ST28.

In step ST26, the output unit 82D displays the size 118 of the lesion 42 in the first display region 36 of the screen 35 (see FIG. 9). After the processing of step ST26 is performed, the medical support process proceeds to step ST28.

In step ST28, the control unit 82B determines whether a condition for ending the medical support process is satisfied. An example of the condition for ending the medical support process is a condition in which an instruction to end the medical support process is given to the endoscope system 10 (for example, a condition in which the instruction to end the medical support process is received by the reception device 64).

If the condition for ending the medical support process is not satisfied in step ST28, the determination is negative, and the medical support process proceeds to step ST10. If the condition for ending the medical support process is satisfied in step ST28, the determination is affirmative, and the medical support process ends.

As described above, in the endoscope system 10, the measurement output process 116 is performed based on the frame 40 obtained by imaging the intestinal wall 32 including the lesion 42 with the camera 52 inserted into the large intestine 28 of the subject 26. When the measurement output process 116 is performed, the size 118 of the lesion 42 is measured, and the measured size 118 is output. In the endoscope system 10, then, the measurement output process 116 is controlled in accordance with the appearance information related to the appearance of the surface region 32A (see FIG. 7) in the frame 40. Positional relationship identification information that can identify the relative positional relationship between the camera 52 and the surface region 32A is used as the appearance information. The degree of extension 114 is used as the positional relationship identification information. For this reason, in the endoscope system 10, the measurement output process 116 is executed in accordance with the degree of extension 114. That is, the measurement output process 116 is executed when the degree of extension 114 is “low”, and the measurement output process 116 is not executed when the degree of extension 114 is “high”.

Accordingly, the endoscope system 10 enables the doctor 12 to grasp the size 118 of the lesion 42 in a case where the appearance of the surface region 32A in the frame 40 is correct (that is, appropriate). In other words, the doctor 12 can grasp the size 118 of the lesion 42 in a case where the relative positional relationship between the camera 52 and the surface region 32A is correct (that is, appropriate). Furthermore, according to the endoscope system 10, the degree of extension 114 is used as information that can identify the relative positional relationship between the camera 52 and the surface region 32A, which can contribute to the accurate determination of whether the relative positional relationship between the camera 52 and the surface region 32A is correct (that is, appropriate).

In the endoscope system 10, furthermore, the lesion 42 of which the size 118 is to be measured through the measurement output process 116 is recognized from the frame 40 by performing the recognition process 96 on the frame 40. Accordingly, the endoscope system 10 enables recognition of the lesion 42 (in other words, identification of the lesion 42) of which the size 118 is to be measured through the measurement output process 116 without involving the doctor 12.

In the endoscope system 10, furthermore, the size 118 of the lesion 42 is measured, and the measured size 118 is displayed in the first display region 36 of the screen 35. Accordingly, the endoscope system 10 enables the doctor 12 to visually recognize the size 118 of the lesion 42 in a case where the appearance of the surface region 32A in the frame 40 is correct (that is, appropriate).

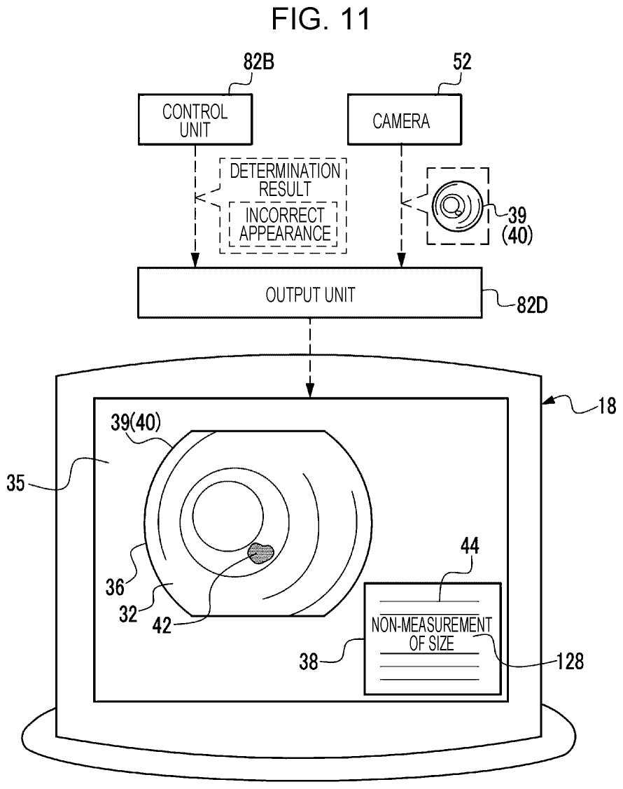

While the embodiment described above provides an example embodiment in which the size 118 of the lesion 42 is not measured when the control unit 82B determines that the appearance of the lesion 42 in the frame 40 is incorrect, the technology of the present disclosure is not limited to this. For example, the size 118 of the lesion 42 may be measured when the control unit 82B determines that the appearance of the lesion 42 in the frame 40 is incorrect, although the measured size 118 is not output (that is, the measured size 118 is not displayed). While the embodiment described above provides an example embodiment in which the size 118 of the lesion 42 is not measured when the control unit 82B determines that the appearance of the lesion 42 in the frame 40 is incorrect, in addition to simply not measuring the size 118 of the lesion 42, for example, as illustrated in FIG. 11, the output unit 82D may display size non-measurement information 128 that can identify that the measurement of the size 118 has not been carried out, in the second display region 38 of the screen 35 as the auxiliary information 44. The size non-measurement information 128 is an example of the “first information” according to the technology of the present disclosure. The size non-measurement information 128 may be text information that can identify that the measurement of the size 118 has not been carried out, or may be an image or the like that can identify that the measurement of the size 118 has not been carried out. The size non-measurement information 128 may be displayed in a display region other than the second display region 38 (for example, the first display region 36 and/or a screen other than the screen 35).

As described above, the size non-measurement information 128 displayed in the second display region 38 allows the doctor 12 to grasp that the measurement of the size 118 has not been carried out.

While the display of the size non-measurement information 128 is illustrated as an example of the output of the size non-measurement information 128, this is merely an example. For example, the size non-measurement information 128 may be output as audio, may be recorded on a medium (for example, a sheet) by a printer, may be stored in a storage medium (for example, a memory, a magnetic tape, and/or the like), or may be written in an electronic medical record.

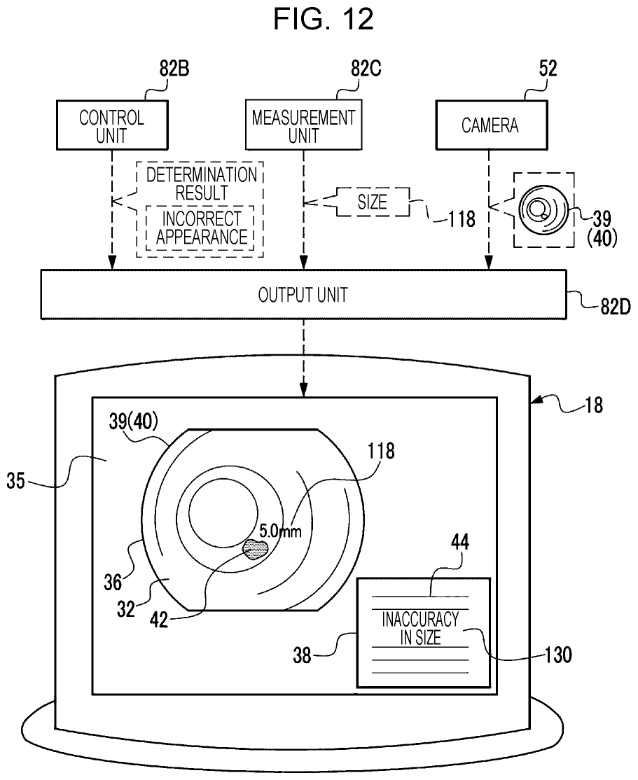

While the embodiment described above provides an example embodiment in which the size 118 is not displayed on the screen 35 when the control unit 82B determines that the appearance of the lesion 42 in the frame 40 is incorrect, this is merely an example. For example, as illustrated in FIG. 12, even if the control unit 82B determines that the appearance of the lesion 42 in the frame 40 is incorrect, the size 118 may be displayed on the screen 35 (in the example illustrated in FIG. 12, in the first display region 36 of the screen 35). In this case, the output unit 82D displays size inaccuracy information 130 that can identify that the size 118 displayed on the screen 35 is inaccurate, in the second display region 38 of the screen 35 as the auxiliary information 44. The size inaccuracy information 130 is an example of the “second information” according to the technology of the present disclosure. The size inaccuracy information 130 may be text information that can identify that the size 118 displayed on the screen 35 is inaccurate, or may be an image or the like that can identify that the size 118 displayed on the screen 35 is inaccurate. The size inaccuracy information 130 may be displayed in a display region other than the second display region 38 (for example, the first display region 36 and/or a screen other than the screen 35). Alternatively, the size inaccuracy information 130 may be displayed in a manner that can identify that the size inaccuracy information 130 is associated with the size 118. For example, the size inaccuracy information 130 is displayed in the first display region 36 so as to be adjacent to the size 118.

As described above, the size inaccuracy information 130 displayed on the screen 35 allows the doctor 12 to grasp that the size 118 displayed on the screen 35 is inaccurate.

While the display of the size inaccuracy information 130 is illustrated as an example of the output of the size inaccuracy information 130, this is merely an example. For example, the size inaccuracy information 130 may be output as audio, may be recorded on a medium (for example, a sheet) by a printer, may be stored in a storage medium (for example, a memory, a magnetic tape, and/or the like), or may be written in an electronic medical record.

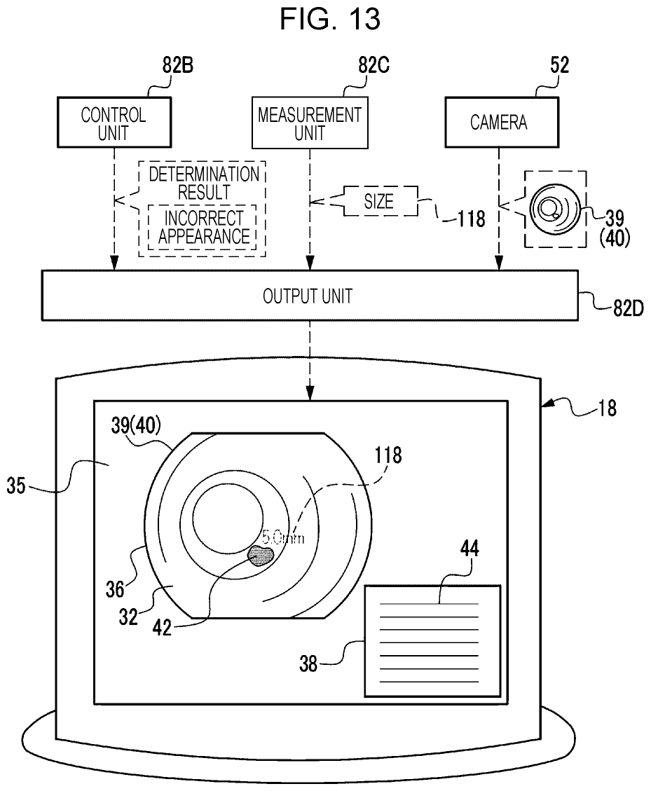

While the embodiment described above provides an example embodiment in which the size 118 is not displayed on the screen 35 when the control unit 82B determines that the appearance of the lesion 42 in the frame 40 is incorrect, for example, as illustrated in FIG. 13, the output unit 82D may display the size 118 on the screen 35 (in the example illustrated in FIG. 13, in the first display region 36 of the screen 35) even if the control unit 82B determines that the appearance of the lesion 42 in the frame 40 is incorrect. In this case, the output unit 82D performs a perception level reduction process as one of the processes included in the measurement output process 116. The perception level reduction process refers to a process for reducing the level at which the display of the size 118 in the first display region 36 in a case where the control unit 82B determines that the appearance of the lesion 42 in the frame 40 is incorrect is visually perceived by the doctor 12, such that the level becomes lower than a reference perception level. An example of the reference perception level is a level at which the display of the size 118 in the first display region 36 in a case where the control unit 82B determines that the appearance of the lesion 42 in the frame 40 is correct is visually perceived by the doctor 12.

Examples of a method for reducing the level at which the display of the size 118 is visually perceived by the doctor 12 include a method for making the display of the size 118 semi-transparent by using an alpha blending method, and a method for making the text representing the size 118 less highlighted than the text representing the size 118 at the reference perception level in terms of the stroke thickness of the text representing the size 118, the stroke color of the text representing the size 118, the stroke luminance of the text representing the size 118, the stroke density of the text representing the size 118, and/or the like.

As described above, when the control unit 82B determines that the appearance of the lesion 42 in the frame 40 is incorrect, the perception level reduction process is performed as one of the processes included in the measurement output process 116. This enables the doctor 12 to grasp that the size 118 displayed in the first display region 36 is inaccurate.

While the embodiment described above provides an example embodiment in which the control unit 82B determines whether the appearance of the lesion 42 in the frame 40 is correct, based on the degree of extension 114, the technology of the present disclosure is not limited to this. For example, instead of the degree of extension 114, an angle formed by an optical axis OA (see FIG. 7) of the camera 52 and the surface region 32A (see FIG. 7) (hereinafter, also simply referred to as the “angle”) may be used as the positional relationship identification information. In this case, it is sufficient that the appearance of the lesion 42 in the frame 40 be determined to be incorrect when the angle falls within a reference angle range (for example, an orthogonal state or a near-orthogonal state) and that the appearance of the lesion 42 in the frame 40 be determined to be correct when the angle is less than a reference angle range. The reference angle range refers to, for example, an angle range determined as an allowable range of angles within which the size 118 of the lesion 42 can be accurately measured. The upper limit value and the lower limit value that define the reference angle range may be fixed values or variable values that are changed in accordance with an instruction received by the reception device 64 and/or various conditions.

As described above, an angle is used as the positional relationship identification information, which can contribute to the accurate determination of whether the relative positional relationship between the camera 52 and the surface region 32A is correct, as in the embodiment described above.

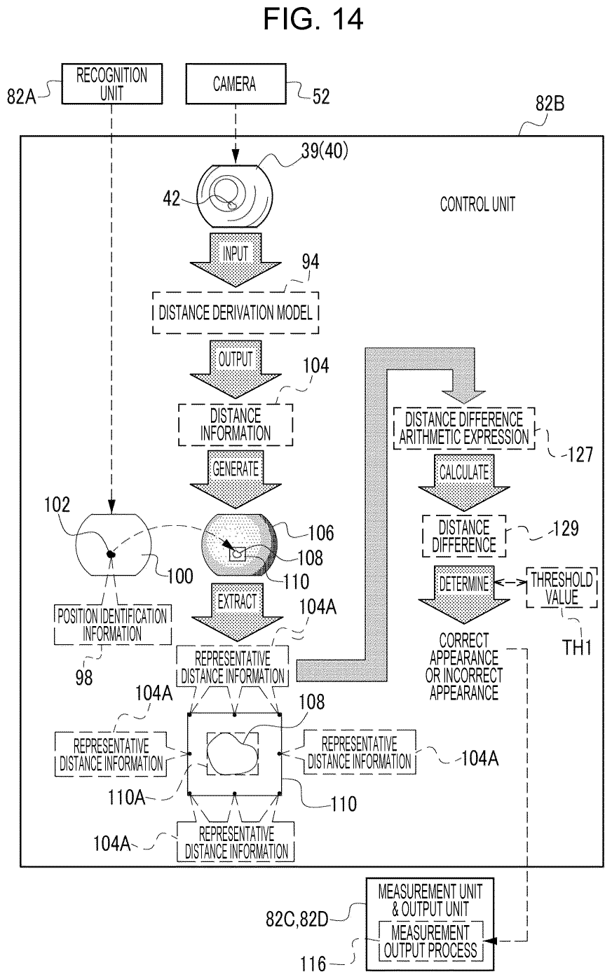

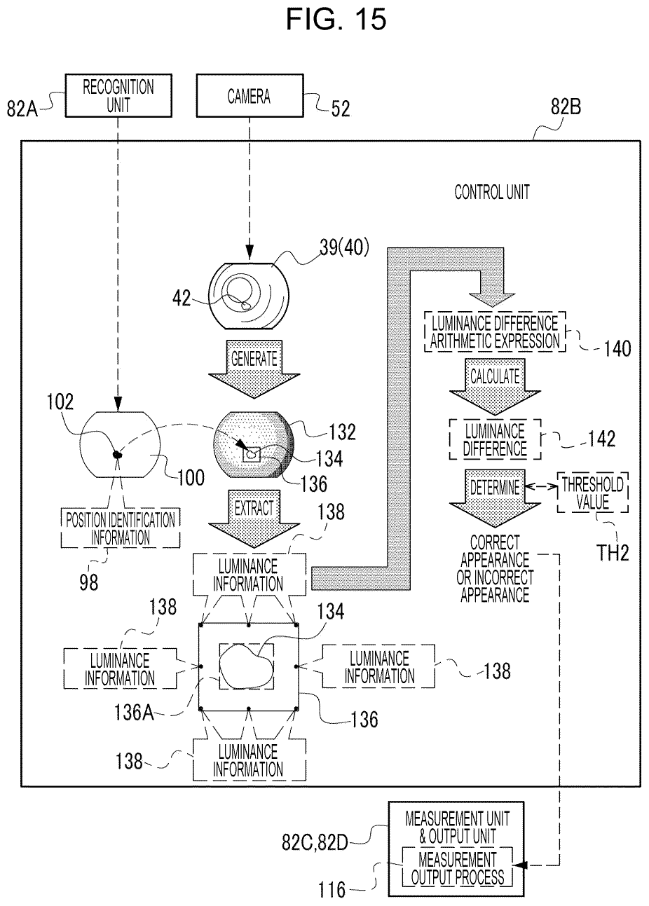

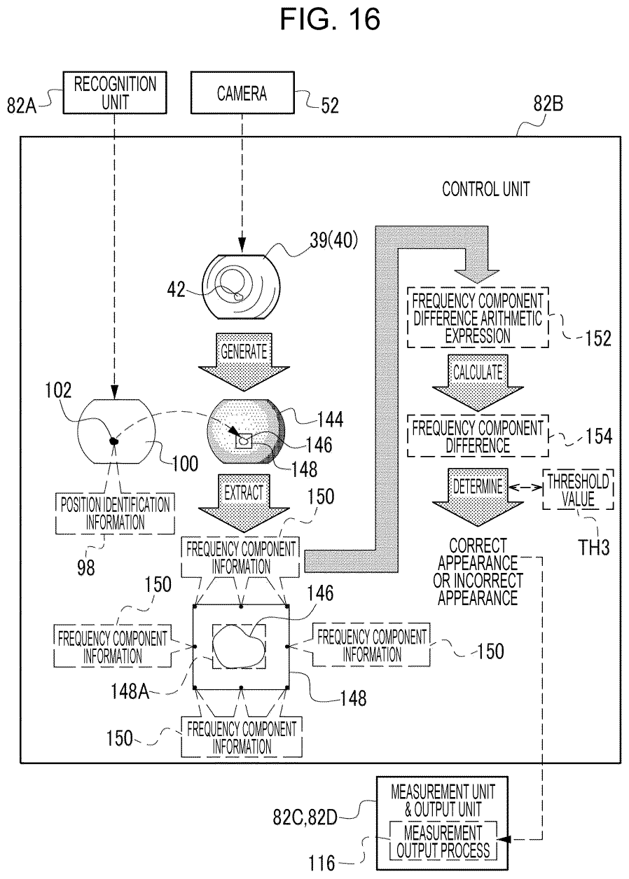

While the embodiment described above provides an example embodiment in which the control unit 82B determines whether the appearance of the lesion 42 in the frame 40 is correct, based on the degree of extension 114, the technology of the present disclosure is not limited to this. For example, as illustrated in FIGS. 14 to 16, instead of or together with the degree of extension 114, the angle described above, a distance difference 129 (see FIG. 14), a luminance difference 142 (see FIG. 15), and/or a frequency component difference 154 (see FIG. 16) may be used as the positional relationship identification information. The angle is an example of the “angle” according to the technology of the present disclosure, the distance difference 129 is an example of the “first depth” according to the technology of the present disclosure, the luminance difference 142 is an example of the “degree of luminance difference” according to the technology of the present disclosure, and the frequency component difference 154 is an example of the “degree of frequency component difference” according to the technology of the present disclosure.

The distance difference 129 illustrated in FIG. 14 refers to a difference in distance between a plurality of locations in the surface region 32A within the frame 40 (that is, a difference in depth in the depth direction between the plurality of locations in the surface region 32A). Examples of the distance difference 129 include the absolute value of the difference between the maximum value and the minimum value of the plurality of pieces of representative distance information 104A.

As an example, as illustrated in FIG. 14, the control unit 82B calculates the distance difference 129 by using a distance difference arithmetic expression 127. The distance difference arithmetic expression 127 is an arithmetic expression in which the plurality of pieces of representative distance information 104A are dependent variables and the distance difference 129 is an independent variable. The control unit 82B inputs the plurality of pieces of representative distance information 104A extracted from the partial distance image 110 to the distance difference arithmetic expression 127. The distance difference arithmetic expression 127 outputs a distance difference 129 corresponding to the plurality of pieces of representative distance information 104A that have been input.

The control unit 82B determines whether the appearance of the lesion 42 in the frame 40 is correct in accordance with the distance difference 129. For example, the control unit 82B compares the distance difference 129 with a threshold value TH1, determines that the appearance of the lesion 42 in the frame 40 is incorrect when the distance difference 129 is greater than or equal to the threshold value TH1, and determines that the appearance of the lesion 42 in the frame 40 is correct when the distance difference 129 is less than the threshold value TH1. As in the embodiment described above, the control unit 82B controls the measurement output process 116 in accordance with the appearance of the lesion 42 in the frame 40. For example, the measurement output process 116 is not performed when the appearance of the lesion 42 in the frame 40 is incorrect, and the measurement output process 116 is performed when the appearance of the lesion 42 in the frame 40 is correct. The threshold value TH1 refers to, for example, a value determined as an upper limit value of the distance difference 129 at which the size 118 of the lesion 42 can be accurately measured. The threshold value TH1 may be a fixed value or a variable value that is changed in accordance with an instruction received by the reception device 64 and/or various conditions.

As described above, in the example illustrated in FIG. 14, the distance difference 129 is used as information that can identify the relative positional relationship between the camera 52 and the surface region 32A, which can contribute to the accurate determination of whether the relative positional relationship between the camera 52 and the surface region 32A is correct, as in the embodiment described above.

While the distance difference 129 is exemplified here, this is merely an example, and a distance ratio may be used. The distance ratio refers to, for example, the ratio of the maximum value among the plurality of pieces of representative distance information 104A to the minimum value among the plurality of pieces of representative distance information 104A.

The luminance difference 142 illustrated in FIG. 15 refers to a difference in luminance between a plurality of locations in the surface region 32A within the frame 40. Since the luminance decreases as the distance from the camera 52 along the optical axis OA (see FIG. 7) in the depth direction of the large intestine 28 increases, it is determined here whether the relative positional relationship between the camera 52 and the surface region 32A is correct (for example, whether the camera 52 directly faces the surface region 32A), based on the luminance difference 142.

As an example, as illustrated in FIG. 15, the control unit 82B generates a luminance image 132, based on the frame 40. The luminance image 132 is an image obtained by mapping the luminance on a pixel-by-pixel basis in the frame 40.

The control unit 82B extracts luminance information 138 indicating the luminance from a partial luminance image 136 determined based on a segmentation-corresponding region 134 in the luminance image 132 in a manner similar to that for the extraction of the representative distance information 104A from the partial distance image 110 in the embodiment described above.

The segmentation-corresponding region 134 is a closed region corresponding to the position of the segmentation image 102 in the luminance image 132. The partial luminance image 136 is determined based on the segmentation-corresponding region 134.

For example, the partial luminance image 136 is a luminance image cropped from the luminance image 132 using a rectangular frame obtained by enlarging a rectangular frame 136A circumscribing the segmentation-corresponding region 134 by the predetermined magnification described in the embodiment described above.

The luminance information 138 is luminance information obtained from each of a plurality of locations in the partial luminance image 136. The plurality of locations are at least two locations including one location and the other location between which the segmentation-corresponding region 134 is crossed. Examples of crossing the segmentation-corresponding region 134 include crossing the segmentation-corresponding region 134 by the longest distance and crossing the segmentation-corresponding region 134 by the shortest distance.

In the example illustrated in FIG. 15, luminance information 138 obtained from each of eight locations in the partial luminance image 136 is exemplified. The eight locations in the partial luminance image 136 are the same as the eight locations in the partial distance image 110 described in the embodiment described above. This is merely an example, and it is sufficient to use pieces of luminance information 138 for a plurality of locations (here, as an example, eight locations) that can identify the extent to which the lesion 42 extends to the side deeper into the body cavity (that is, in the depth direction of the large intestine 28) when the lesion 42 is viewed from the camera 52 (in other words, the degree to which the camera 52 directly faces the lesion 42).

The control unit 82B determines whether the appearance of the lesion 42 in the frame 40 is correct, based on the luminance difference 142. Examples of the luminance difference 142 include the absolute value of the difference between the maximum value and the minimum value of the plurality of pieces of luminance information 138.

The control unit 82B calculates the luminance difference 142 by using a luminance difference arithmetic expression 140. The luminance difference arithmetic expression 140 is an arithmetic expression in which the plurality of pieces of luminance information 138 are dependent variables and the luminance difference 142 is an independent variable. The control unit 82B inputs the plurality of pieces of luminance information 138 extracted from the partial luminance image 136 to the luminance difference arithmetic expression 140. The luminance difference arithmetic expression 140 outputs a luminance difference 142 corresponding to the plurality of pieces of luminance information 138 that have been input.

The control unit 82B determines whether the appearance of the lesion 42 in the frame 40 is correct in accordance with the luminance difference 142. For example, the control unit 82B compares the luminance difference 142 with a threshold value TH2, determines that the appearance of the lesion 42 in the frame 40 is incorrect when the luminance difference 142 is greater than or equal to the threshold value TH2, and determines that the appearance of the lesion 42 in the frame 40 is correct when the luminance difference 142 is less than the threshold value TH2. As in the embodiment described above, the control unit 82B controls the measurement output process 116 in accordance with the appearance of the lesion 42 in the frame 40. The threshold value TH2 refers to, for example, a value determined as an upper limit value of the luminance difference 142 at which the size 118 of the lesion 42 can be accurately measured. The threshold value TH2 may be a fixed value or a variable value that is changed in accordance with an instruction received by the reception device 64 and/or various conditions.

As described above, in the example illustrated in FIG. 15, the luminance difference 142 is used as information that can identify the relative positional relationship between the camera 52 and the surface region 32A, which can contribute to the accurate determination of whether the relative positional relationship between the camera 52 and the surface region 32A is correct, as in the embodiment described above.

While the luminance difference 142 is exemplified here, this is merely an example, and a luminance ratio may be used. The luminance ratio refers to, for example, the ratio of the maximum value among the plurality of pieces of luminance information 138 to the minimum value among the plurality of pieces of luminance information 138.

The frequency component difference 154 illustrated in FIG. 16 refers to a difference in frequency component between a plurality of locations in the surface region 32A within the frame 40. Since the frequency component within the depth of field of the camera 52 is higher than the frequency component outside the depth of field of the camera 52, it is determined here whether the relative positional relationship between the camera 52 and the surface region 32A is correct (for example, whether the camera 52 directly faces the surface region 32A), based on the frequency component difference 154.

As an example, as illustrated in FIG. 16, the control unit 82B generates a frequency component image 144, based on the frame 40. The frequency component image 144 is an image obtained by mapping the frequency component on a pixel-by-pixel basis in the frame 40.

The control unit 82B extracts frequency component information 150 indicating the frequency component from a partial frequency component image 148 determined based on a segmentation-corresponding region 146 in the frequency component image 144 in a manner similar to that for the extraction of the representative distance information 104A from the partial distance image 110 in the embodiment described above.

The segmentation-corresponding region 146 is a closed region corresponding to the position of the segmentation image 102 in the frequency component image 144. The partial frequency component image 148 is determined based on the segmentation-corresponding region 146.

For example, the partial frequency component image 148 is a frequency component image cropped from the frequency component image 144 using a rectangular frame obtained by enlarging a rectangular frame 148A circumscribing the segmentation-corresponding region 146 by the predetermined magnification described in the embodiment described above.

The frequency component information 150 is frequency component information obtained from each of a plurality of locations in the partial frequency component image 148. The plurality of locations are at least two locations including one location and the other location between which the segmentation-corresponding region 146 is crossed. Examples of crossing the segmentation-corresponding region 146 include crossing the segmentation-corresponding region 146 by the longest distance and crossing the segmentation-corresponding region 146 by the shortest distance.

In the example illustrated in FIG. 16, frequency component information 150 obtained from each of eight locations in the partial frequency component image 148 is exemplified. The eight locations in the partial frequency component image 148 are the same as the eight locations in the partial distance image 110 described in the embodiment described above. This is merely an example, and it is sufficient to use pieces of frequency component information 150 for a plurality of locations (here, as an example, eight locations) that can identify the extent to which the lesion 42 extends to the side deeper into the body cavity (that is, in the depth direction of the large intestine 28) when the lesion 42 is viewed from the camera 52 (in other words, the degree to which the camera 52 directly faces the lesion 42).

The control unit 82B determines whether the appearance of the lesion 42 in the frame 40 is correct, based on the frequency component difference 154. Examples of the frequency component difference 154 include the absolute value of the difference between the maximum value and the minimum value of the plurality of pieces of frequency component information 150.

The control unit 82B calculates the frequency component difference 154 by using a frequency component difference arithmetic expression 152. The frequency component difference arithmetic expression 152 is an arithmetic expression in which the plurality of pieces of frequency component information 150 are dependent variables and the frequency component difference 154 is an independent variable. The control unit 82B inputs the plurality of pieces of frequency component information 150 extracted from the partial frequency component image 148 to the frequency component difference arithmetic expression 152. The frequency component difference arithmetic expression 152 outputs a frequency component difference 154 corresponding to the plurality of pieces of frequency component information 150 that have been input.

The control unit 82B determines whether the appearance of the lesion 42 in the frame 40 is correct in accordance with the frequency component difference 154. For example, the control unit 82B compares the frequency component difference 154 with a threshold value TH3, determines that the appearance of the lesion 42 in the frame 40 is incorrect when the frequency component difference 154 is greater than or equal to the threshold value TH3, and determines that the appearance of the lesion 42 in the frame 40 is correct when the frequency component difference 154 is less than the threshold value TH3. As in the embodiment described above, the control unit 82B controls the measurement output process 116 in accordance with the appearance of the lesion 42 in the frame 40. The threshold value TH3 refers to, for example, a value determined as an upper limit value of the frequency component difference 154 at which the size 118 of the lesion 42 can be accurately measured. The threshold value TH3 may be a fixed value or a variable value that is changed in accordance with an instruction received by the reception device 64 and/or various conditions.

As described above, in the example illustrated in FIG. 16, the frequency component difference 154 is used as information that can identify the relative positional relationship between the camera 52 and the surface region 32A, which can contribute to the accurate determination of whether the relative positional relationship between the camera 52 and the surface region 32A is correct, as in the embodiment described above.

While the frequency component difference 154 is exemplified here, this is merely an example, and a frequency component ratio may be used. The frequency component ratio refers to, for example, the ratio of the maximum value among the plurality of pieces of frequency component information 150 to the minimum value among the plurality of pieces of frequency component information 150.

The examples illustrated in FIGS. 6 and 14 provide an example embodiment in which the partial distance image 110, which is cropped from the distance image 106 using a rectangular frame obtained by enlarging the rectangular frame 110A circumscribing the segmentation-corresponding region 108 by the predetermined magnification, is used. The example illustrated in FIG. 15 provides an example embodiment in which the partial luminance image 136, which is cropped from the luminance image 132 using a rectangular frame obtained by enlarging the rectangular frame 136A circumscribing the segmentation-corresponding region 134 by the predetermined magnification, is used. The example illustrated in FIG. 16 provides an example embodiment in which the partial frequency component image 148, which is cropped from the frequency component image 144 using a rectangular frame obtained by enlarging the rectangular frame 148A circumscribing the segmentation-corresponding region 146 by the predetermined magnification, is used. However, these are merely examples. For example, in the examples illustrated in FIGS. 6 and 14, an image cropped from the distance image 106 using the rectangular frame 110A circumscribing the segmentation-corresponding region 108 may be used as the partial distance image 110. In the example illustrated in FIG. 15, an image cropped from the luminance image 132 using the rectangular frame 136A circumscribing the segmentation-corresponding region 134 may be used as the partial luminance image 136. In the example illustrated in FIG. 16, an image cropped from the frequency component image 144 using the rectangular frame 148A circumscribing the segmentation-corresponding region 146 may be used as the partial frequency component image 148.

In addition, the control unit 82B may determine whether the appearance of the lesion 42 in the frame 40 is correct, based on two or more measures among the degree of extension 114, the angle, the distance difference 129, the luminance difference 142, and the frequency component difference 154. In this case, for example, as represented by Equation (1) below, the measure to be emphasized may be defined based on weighting.

(Total determination value)=(degree of extension)×A+(angle)×B+(distance difference)×C+(luminance difference)×D+(frequency component difference)×E (1)

In Equation (1) above, A to E are weights. The total determination value is a value to be compared with a reference determination value. When the “total determination value≥reference determination value” is satisfied, it is determined that the appearance of the lesion 42 in the frame 40 is incorrect. When the “total determination value<reference determination value” is satisfied, it is determined that the appearance of the lesion 42 in the frame 40 is correct. The weights A to E and/or the reference determination value may be fixed values or variable values that are changed in accordance with an instruction received by the reception device 64 and/or various conditions.

While the degree of extension 114, the angle, the distance difference 129, the luminance difference 142, and the frequency component difference 154 are illustrated as examples to be used as the appearance information, the technology of the present disclosure is not limited to this. The information to be used as the appearance information may be, for example, the amount of blur in an image region including the lesion 42 appearing in the frame 40, the amount of artifact in the image region including the lesion 42 appearing in the frame 40, and/or the like.

For example, in a case where the information to be used as the appearance information is the amount of blur in the image region including the lesion 42 appearing in the frame 40, the control unit 82B determines that the appearance of the lesion 42 in the frame 40 is incorrect when the amount of blur exceeds a reference amount of blur (for example, an amount of blur determined as the upper limit value of the range of amounts of blur at which the recognition unit 82A can accurately recognize the lesion 42).

For example, in a case where the information to be used as the appearance information is the amount of artifact in the image region including the lesion 42 appearing in the frame 40, the control unit 82B determines that the appearance of the lesion 42 in the frame 40 is incorrect when the amount of artifact exceeds a reference amount of artifact (for example, an amount of artifact determined as the upper limit value of the range of amounts of artifact at which the recognition unit 82A can accurately recognize the lesion 42).

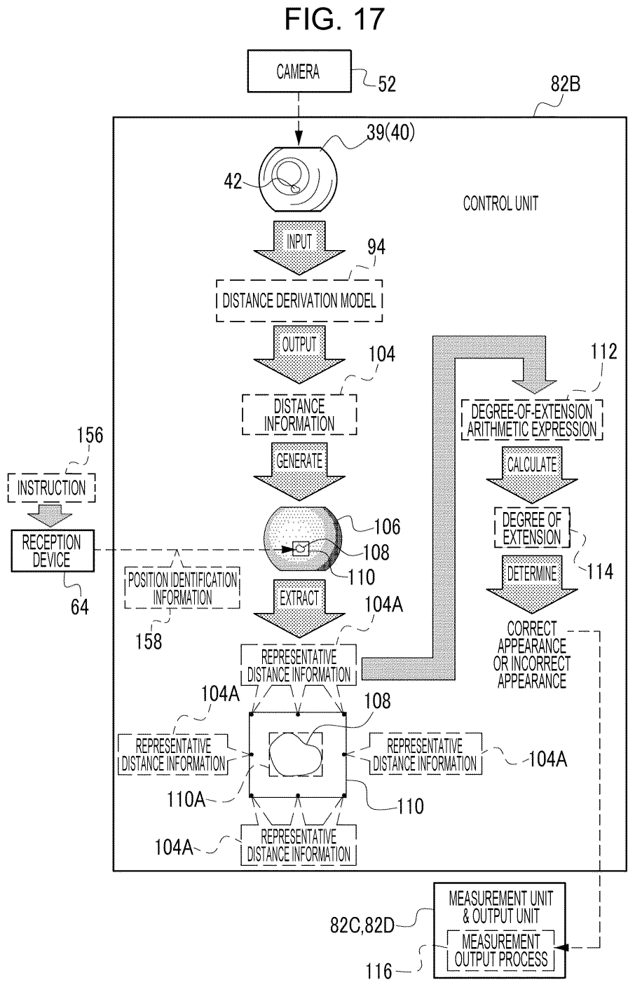

While the embodiment described above provides an example embodiment in which the segmentation-corresponding region 108 is identified from the position identification information 98 obtained through the recognition process 96 performed on the frame 40, the technology of the present disclosure is not limited to this. For example, as illustrated in FIG. 17, the segmentation-corresponding region 108 may be identified in accordance with an instruction 156 given by the doctor 12. In the example illustrated in FIG. 17, the instruction 156 is received by the reception device 64. The instruction 156 includes position identification information 158 corresponding to the position identification information 98 described in the embodiment described above, and the control unit 82B identifies the segmentation-corresponding region 108 from the distance image 106 in accordance with the position identification information 158. Thus, the lesion 42 located at a position intended by the doctor 12 can be used as the lesion 42 of which the size 118 is measured through the measurement output process 116. In the example illustrated in FIG. 17, the instruction 156 is an example of the “instruction” according to the technology of the present disclosure.

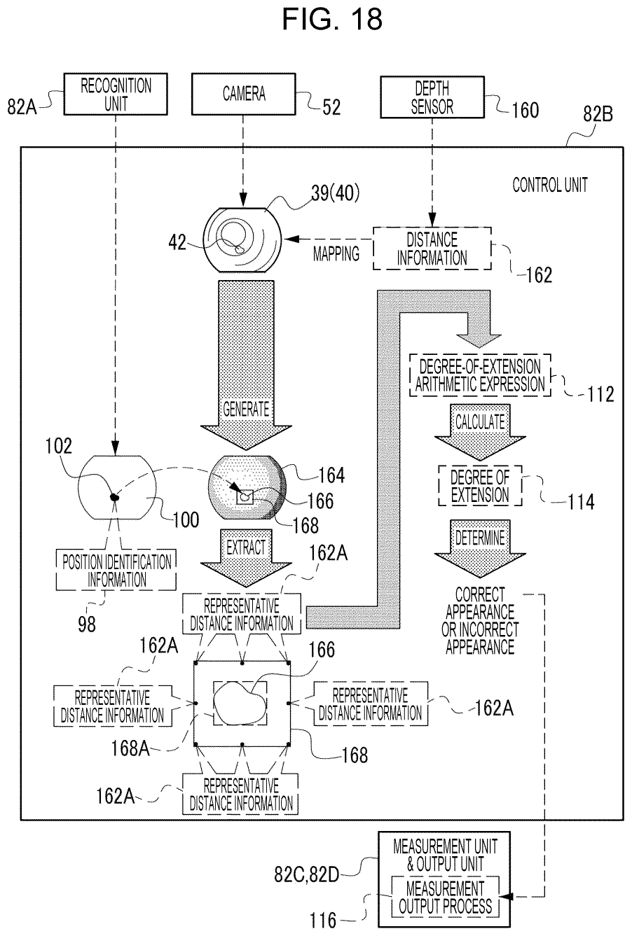

While the embodiment described above provides an example embodiment in which the control unit 82B generates the distance image 106 (see FIG. 6) from the frame 40 by using the distance derivation model 94 (see FIG. 6), the technology of the present disclosure is not limited to this. For example, as illustrated in FIG. 18, a depth sensor 160 (for example, a sensor that measures a distance by a laser ranging method, a phase difference method, and/or the like) may measure the depth of the large intestine 28 in the depth direction, and the control unit 82B may generate a distance image 164, based on the measured depth. For example, the depth sensor 160 is disposed in the tip portion 50 (see FIG. 2). In the example illustrated in FIG. 18, the depth sensor 160 is an example of the “depth sensor” according to the technology of the present disclosure.

The distance image 164 is a distance image corresponding to the distance image 106 (see FIG. 6) described in the embodiment described above. In the example illustrated in FIG. 18, distance information 162 corresponding to the distance information 104 (see FIG. 6) described in the embodiment described above is mapped to the frame 40 on a pixel-by-pixel basis to generate the distance image 164. The distance information 162 is information indicating a distance obtained by the depth sensor 160 measuring the distance of the large intestine 28 in the depth direction.

In the example illustrated in FIG. 18, a segmentation-corresponding region 166, a rectangular frame 168A, and a partial distance image 168 are illustrated. The segmentation-corresponding region 166 corresponds to the segmentation-corresponding region 108 (see FIG. 6) described in the embodiment described above and is obtained in a manner similar to that for the segmentation-corresponding region 108. The rectangular frame 168A corresponds to the rectangular frame 110A (see FIG. 6) described in the embodiment described above and is obtained in a manner similar to that for the rectangular frame 110A. The partial distance image 168 corresponds to the partial distance image 110 (see FIG. 6) described in the embodiment described above and is obtained in a manner similar to that for the partial distance image 110.

In the example illustrated in FIG. 18, furthermore, a plurality of pieces of representative distance information 162A are illustrated. The plurality of pieces of representative distance information 162A correspond to the plurality of pieces of representative distance information 104A (see FIG. 6) described in the embodiment described above and are obtained in a manner similar to that for the plurality of pieces of representative distance information 104A. When the plurality of pieces of representative distance information 162A are input to the degree-of-extension arithmetic expression 112, the degree-of-extension arithmetic expression 112 outputs a degree of extension 114 corresponding to the plurality of pieces of input representative distance information 162A. Also in this case, effects similar to those of the embodiment described above can be expected.

In addition, a distance difference corresponding to the distance difference 129 may be calculated from the plurality of pieces of representative distance information 162A in a manner similar to that for the calculation of the distance difference 129 from the plurality of pieces of representative distance information 104A. In this case, effects similar to those of the example illustrated in FIG. 14 can be expected. The distance difference calculated from the plurality of pieces of representative distance information 162A is an example of the “second depth” according to the technology of the present disclosure.

While the embodiment described above provides an example embodiment in which the endoscopic moving image 39 is displayed in the first display region 36, a result of the recognition process 96 performed on the endoscopic moving image 39 may be displayed superimposed on the endoscopic moving image 39 in the first display region 36. At least a portion of the segmentation image 102 obtained as a result of the recognition process 96 performed on the endoscopic moving image 39 may be displayed superimposed on the endoscopic moving image 39. An example in which at least a portion of the segmentation image 102 is displayed superimposed on the endoscopic moving image 39 is an example embodiment in which the outer contour of the segmentation image 102 is displayed superimposed on the endoscopic moving image 39 by using an alpha blending method.

In addition, for example, when the recognition process 96 is performed using an AI-based bounding box method, a bounding box may be displayed superimposed on the endoscopic moving image 39 in the first display region 36. Furthermore, for example, when a plurality of lesions 42 appear in the endoscopic moving image 39, at least a portion of the segmentation image 102 and/or a bounding box is displayed superimposed in the first display region 36 as information that can visually identify which of the lesions 42 the measured size 118 corresponds to. Alternatively, the probability map 100 and/or a bounding box for the lesion 42 corresponding to the measured size 118 may be displayed in a display region different from the first display region 36. Alternatively, for example, the probability map 100 may be displayed superimposed on the endoscopic moving image 39 in the first display region 36. The information displayed superimposed on the endoscopic moving image 39 may be information depicted semi-transparent (for example, information subjected to alpha blending).

While the embodiment described above provides an example embodiment in which the length, in real space, of the longest range across the lesion 42 along the line segment 122 is measured as the size 118, the technology of the present disclosure is not limited to this. For example, the length, in real space, of the range corresponding to the longest line segment parallel to the short sides of the rectangular frame 124 for the image region indicating the lesion 42 may be measured as the size 118 and displayed on the screen 35. In this case, the doctor 12 can grasp the length, in real space, of the longest range across the lesion 42 along the longest line segment parallel to the short sides of the rectangular frame 124 for the image region indicating the lesion 42.

Furthermore, the size of the lesion 42 in real space in relation to the radius and/or diameter of a circumcircle of the image region indicating the lesion 42 may be measured and displayed on the screen 35. In this case, the doctor 12 can grasp the size of the lesion 42 in real space in relation to the radius and/or diameter of the circumcircle of the image region indicating the lesion 42.

While the embodiment described above provides an example embodiment in which the size 118 is displayed in the first display region 36, this is merely an example. The size 118 may be displayed outside the first display region 36 in a pop-up manner from within the first display region 36, or the size 118 may be displayed in a region other than the first display region 36 on the screen 35. The type of lesion, the category of lesion, and/or the like may also be displayed in the first display region 36 and/or the second display region 38, or may be displayed on a screen other than the screen 35.

The embodiment described above provides an example embodiment in which the size of one lesion 42 is measured and the measurement result is presented to the doctor 12. When a plurality of lesions 42 appear in the frame 40, it is sufficient that the medical support process be executed on each of the plurality of lesions 42. In this case, a mark or the like may be added to an image region of a lesion 42 corresponding to information (size, category, type, and width) displayed on the screen 35 to allow the identification of which of the lesions 42 the information displayed on the screen 35 corresponds to.

While the embodiment described above provides an example embodiment in which the size 118 is measured frame by frame, this is merely an example, and the size 118 may be measured in units of multiple frames.

In the embodiment described above, the AI-based object recognition process is exemplified as the recognition process 96. However, the technology of the present disclosure is not limited to this. The recognition unit 82A may recognize the lesion 42 appearing in the frame 40 by the execution of a non-AI-based object recognition process (for example, template matching or the like).

While the embodiment described above describes an example embodiment in which the arithmetic expression 126 is used to calculate the size 118, the technology of the present disclosure is not limited to this. The size 118 may be measured by performing an AI-based process on the frame 40. In this case, for example, a trained model is used that, in response to an input of the frame 40 including the lesion 42, outputs the size 118 of the lesion 42. To generate the trained model, deep learning is performed on a neural network by using training data in which lesions appearing in images used as example data are assigned annotations indicating the sizes of the lesions as ground-truth data.

While the embodiment described above describes an example embodiment in which the distance information 104 is derived using the distance derivation model 94, the technology of the present disclosure is not limited to this. Other methods for deriving the distance information 104 using an AI-based method include, for example, a method for combining segmentation and depth estimation (for example, regression learning to provide the distance information 104 to the entire image (for example, all the pixels constituting the image) or unsupervised learning to learn the distance of the entire image in an unsupervised way).

While the embodiment described above exemplifies the endoscopic moving image 39, the technology of the present disclosure is not limited to this. The technology of the present disclosure is also applicable to a medical moving image (for example, a moving image obtained by a modality other than the endoscope system 10, such as a radiographic moving image or an ultrasound moving image) other than the endoscopic moving image 39.

While the embodiment described above provides an example embodiment in which the size 118 of the lesion 42 appearing in a moving image is measured, this is merely an example. The technology of the present disclosure is also applicable to the measurement of the size 118 of the lesion 42 appearing in a stop-motion image or a still image.

While the embodiment described above provides an example embodiment in which the distance information 104 extracted from the distance image 106 is input to the arithmetic expression 126, the technology of the present disclosure is not limited to this. For example, it is sufficient that the distance information 104 corresponding to the position identified from the position identification information 98 be extracted from among all the pieces of distance information 104 output from the distance derivation model 94, without the generation of the distance image 106, and the extracted distance information 104 be input to the arithmetic expression 126.

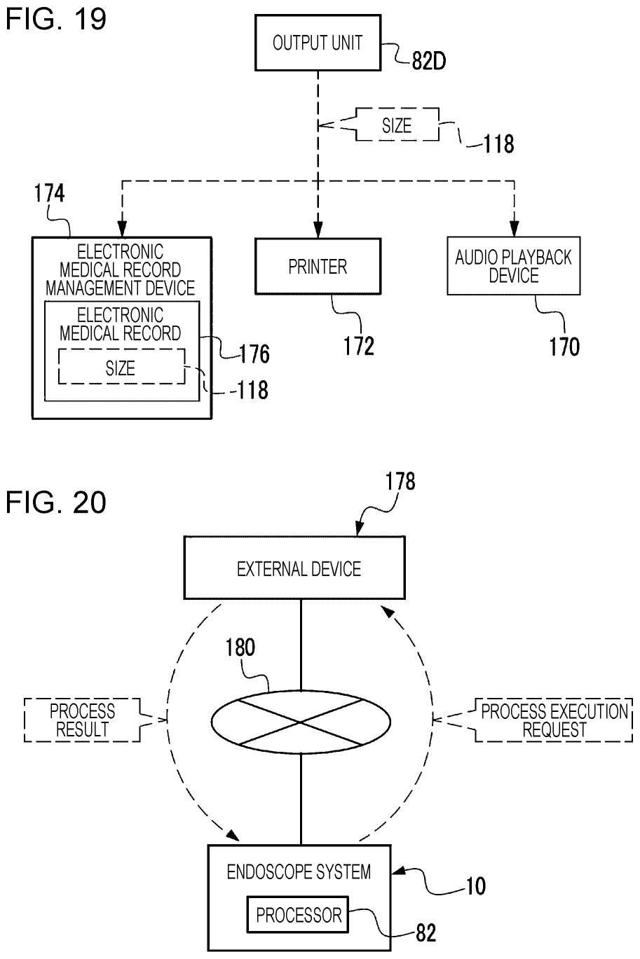

In the examples described above, the size 118 and the like are output to the display device 18, by way of example. However, the technology of the present disclosure is not limited to this, and various kinds of information such as the size 118 (hereinafter referred to as “various kinds of information”) may be output to a device other than the display device 18. As an example, as illustrated in FIG. 19, the various kinds of information may be output to an audio playback device 170, a printer 172, an electronic medical record management device 174, and/or the like as a destination.

The various kinds of information may be output as audio by the audio playback device 170. The various kinds of information may be printed as text or the like on a medium (for example, a sheet) or the like by the printer 172. The various kinds of information may be stored in an electronic medical record 176 managed by the electronic medical record management device 174.

While the examples described above describe an example embodiment in which the various kinds of information are displayed on the screen 35 or the various kinds of information are not displayed on the screen 35, the display of the various kinds of information on the screen 35 means the display of the various kinds of information in a manner perceptible to the user or the like (for example, the doctor 12). The concept that the various kinds of information are not displayed on the screen 35 also includes a concept of reducing the display level of the various kinds of information (for example, the level perceived through the display). For example, the concept that the various kinds of information are not displayed on the screen 35 also includes a concept of displaying the various kinds of information in a display style that does not allow the user or the like to visually perceive the various kinds of information. Examples of the display style in this case include display styles in which the various kinds of information are displayed in a reduced font size, the various kinds of information are depicted as thin lines, the various kinds of information are depicted as broken lines, the various kinds of information blink, the various kinds of information are displayed for an imperceptible period of display time, and the various kinds of information are made transparent to an imperceptible level. The same applies to the various outputs described above, such as audio output, printing, and storage.

While the embodiment described above provides an example embodiment in which the medical support process is performed by the processor 82 included in the endoscope system 10, the technology of the present disclosure is not limited to this, and a device external to the endoscope system 10 may perform at least some of the processing operations included in the medical support process.

In this case, for example, as illustrated in FIG. 20, an external device 178 connected to the endoscope system 10 in a communicable manner via a network 180 (for example, a WAN, a LAN, and/or the like) is used.

Examples of the external device 178 include at least one server that transmits and receives data to and from the endoscope system 10 directly or indirectly via the network 180. The external device 178 receives a process execution instruction given from the processor 82 of the endoscope system 10 via the network 180. Then, the external device 178 executes a process corresponding to the received process execution instruction, and transmits a process result to the endoscope system 10 via the network 180. In the endoscope system 10, the processor 82 receives the process result transmitted from the external device 178 via the network 180 and executes a process using the received process result.

Examples of the process execution instruction include an instruction to cause the external device 178 to execute at least a portion of the medical support process. A first example of at least a portion of the medical support process (that is, a process to be executed by the external device 178) is the recognition process 96. In this case, the external device 178 executes the recognition process 96 in accordance with the process execution instruction given from the processor 82 of the endoscope system 10 via the network 180, and transmits a recognition process result (for example, the position identification information 98, the probability map 100, and/or the like) to the endoscope system 10 via the network 180. In the endoscope system 10, the processor 82 receives the recognition process result and executes a process similar to that in the embodiment described above by using the received recognition process result.

A second example of at least a portion of the medical support process (that is, a process to be executed by the external device 178) is a process performed by the measurement unit 82C. The process performed by the measurement unit 82C refers to, for example, a process for measuring the size 118 of the lesion 42. In this case, the external device 178 executes the process performed by the measurement unit 82C in accordance with the process execution instruction given from the processor 82 of the endoscope system 10 via the network 180, and transmits a measurement process result (for example, the size 118 or the like) to the endoscope system 10 via the network 180. In the endoscope system 10, the processor 82 receives the measurement process result and executes a process similar to that in the embodiment described above by using the received measurement process result.

The process in which the processor 82 causes the external device 178 to measure the size 118 of the lesion 42 and acquires a measurement result (that is, the size 118) from the external device 178 via the network 180 is included in the concept that the processor 82 measures the size 118 of the lesion 42 (that is, the concept of “measurement of the size of the observation target region” according to the technology of the present disclosure).

A third example of at least a portion of the medical support process (that is, a process to be executed by the external device 178) is a process of controlling the measurement output process 116 in accordance with the appearance information. In this case, the external device 178 executes a process of controlling the measurement output process 116 in accordance with the appearance information, in accordance with the process execution instruction given from the processor 82 of the endoscope system 10 via the network 180, receives a control process result, and executes a process similar to that in the embodiment described above (for example, a process of controlling the content to be displayed on the screen 35) by using the received control process result.

A process in which the processor 82 causes the external device 178 to perform a process of controlling the measurement output process 116 in accordance with the appearance information is included in the concept that the processor 82 performs the process of controlling the measurement output process 116 in accordance with the appearance information (that is, the concept of “controlling the measurement output process in accordance with the appearance information” according to the technology of the present disclosure).

For example, the external device 178 is implemented by cloud computing. The cloud computing is merely an example, and the external device 178 may be implemented by network computing such as fog computing, edge computing, or grid computing. Instead of the server, at least one personal computer or the like may be used as the external device 178. Alternatively, the external device 178 may be an arithmetic device with a communication function mounted with a plurality of types of AI functions.

While the embodiment described above provides an example embodiment in which the medical support program 90 is stored in the NVM 86, the technology of the present disclosure is not limited to this. For example, the medical support program 90 may be stored in a portable non-transitory computer-readable storage medium such as an SSD or a USB memory. The medical support program 90 stored in the non-transitory storage medium is installed in the computer 78 of the endoscope system 10. The processor 82 executes the medical support process in accordance with the medical support program 90.

Alternatively, the medical support program 90 may be stored in a storage device of another computer, a server, or the like connected to the endoscope system 10 via a network, and the medical support program 90 may be downloaded in response to a request from the endoscope system 10 and installed in the computer 78.

Not all, but a portion, of the medical support program 90 may be stored in a storage device of another computer, a server device, or the like connected to the endoscope system 10, or not all, but a portion, of the medical support program 90 may be stored in the NVM 86.

Examples of a hardware resource that executes the medical support process may include the following various processors. The processors include, for example, a CPU that is a general-purpose processor configured to execute software, that is, a program, to function as a hardware resource that executes the medical support process. The processors further include, for example, a dedicated electric circuit that is a processor having a circuit configuration designed specifically for executing specific processing, such as an FPGA, a PLD, or an ASIC. Each of the processors incorporates or is connected to a memory, and uses the memory to execute the medical support process.

The hardware resource that executes the medical support process may be configured as one of the various processors or as a combination of two or more processors of the same type or different types (for example, a combination of a plurality of FPGAs or a combination of a CPU and an FPGA). The hardware resource that executes the medical support process may be a single processor.

Examples of configuring the hardware resource as a single processor include, first, a form in which a single processor is configured as a combination of one or more CPUs and software and the processor functions as a hardware resource that executes the medical support process. The examples include, second, a form in which, as typified by an SoC or the like, a processor is used in which the functions of the entire system including a plurality of hardware resources that execute the medical support process are implemented as one IC chip. As described above, the medical support process is implemented by using one or more of the various processors described above as hardware resources.

More specifically, the hardware structure of these various processors may be an electric circuit in which circuit elements such as semiconductor elements are combined. The medical support process described above is merely an example. Thus, it goes without saying that unnecessary steps may be deleted, new steps may be added, or the processing order may be changed without departing from the gist.

The description and drawings presented above provide detailed descriptions of portions according to the technology of the present disclosure and are merely examples of the technology of the present disclosure. For example, the descriptions related to the configurations, functions, operations, and effects described above are descriptions related to an example of the configurations, functions, operations, and effects of portions according to the technology of the present disclosure. Thus, it goes without saying that unnecessary portions may be deleted or new elements may be added or substituted in the description and drawings presented above without departing from the gist of the technology of the present disclosure. To avoid complexity and facilitate understanding of portions according to the technology of the present disclosure, descriptions related to common general technical knowledge and the like, for which no specific explanation is required to implement the technology of the present disclosure, are omitted in the description and drawings presented above.

As used herein, “A and/or B” is synonymous with “at least one of A or B”. That is, “A and/or B” means only A, only B, or a combination of A and B. In this specification, furthermore, a concept similar to that of “A and/or B” is applied also to the expression of three or more matters in combination with “and/or”.

All publications, patent applications, and technical standards described herein are incorporated herein by reference to the same extent as if each individual publication, patent application, and technical standard were specifically and individually indicated to be incorporated by reference.

What is claimed is:

1. A medical support device comprising a processor,
the processor being configured to:
perform a measurement output process for measuring a size of an observation target region, based on a medical image obtained by imaging an imaging target region including the observation target region with a camera inserted into a body cavity and for outputting the size; and
control the measurement output process in accordance with appearance information related to an appearance of a surface region in the medical image, the surface region being a portion of the imaging target region and including the observation target region.

2. The medical support device according to claim 1, wherein
positional relationship identification information enabling identification of a positional relationship is used as the appearance information, the positional relationship being a relative positional relationship between the camera and the surface region.

3. The medical support device according to claim 2, wherein
a degree of luminance difference between a plurality of locations in the surface region in the medical image is used as the positional relationship identification information.

4. The medical support device according to claim 2, wherein
a degree of frequency component difference between a plurality of locations in the surface region in the medical image is used as the positional relationship identification information.

5. The medical support device according to claim 2, wherein
a first depth of the surface region from the camera side is used as the positional relationship identification information, the first depth being obtained based on the medical image.

6. The medical support device according to claim 2, wherein
a second depth of the surface region from the camera side is used as the positional relationship identification information, the second depth being obtained using a depth sensor.

7. The medical support device according to claim 2, wherein
extension information related to an extension of the surface region in a depth direction from the camera side is used as the positional relationship identification information.

8. The medical support device according to claim 2, wherein
an angle between an optical axis of the camera and the surface region is used as the positional relationship identification information.

9. The medical support device according to claim 2, wherein
the processor is configured to:
perform no measurement of the size and/or no output of the size when the positional relationship is not a predetermined positional relationship; and
perform the measurement output process when the positional relationship is the predetermined positional relationship.

10. The medical support device according to claim 2, wherein
the processor is configured to:
perform no measurement of the size and output first information when the positional relationship is not a predetermined positional relationship, the first information being information enabling identification of the size not being measured; and
perform the measurement output process when the positional relationship is the predetermined positional relationship.

11. The medical support device according to claim 2, wherein
the processor is configured to:
perform the measurement output process regardless of whether the positional relationship is a predetermined positional relationship; and
output second information when the positional relationship is not the predetermined positional relationship, the second information being information enabling identification of the size being inaccurate.

12. The medical support device according to claim 2, wherein
the processor is configured to
perform the measurement output process regardless of whether the positional relationship is a predetermined positional relationship, and
the measurement output process includes a perception level reduction process for reducing a level at which an output of the size in a case where the positional relationship is not the predetermined positional relationship is perceived such that the level becomes lower than a level at which an output of the size in a case where the positional relationship is the predetermined positional relationship is perceived.

13. The medical support device according to claim 1, wherein
the observation target region of which the size is to be measured in the measurement output process is recognized from the medical image through an object recognition process performed on the medical image.

14. The medical support device according to claim 1, wherein
the observation target region of which the size is to be measured in the measurement output process is identified from the medical image in accordance with a provided instruction.

15. The medical support device according to claim 1, wherein
the size is output by displaying the size on a screen.

16. The medical support device according to claim 1, wherein
the medical image is an endoscopic image obtained by imaging the imaging target region with an endoscope.

17. The medical support device according to claim 1, wherein
the observation target region is a lesion.

18. An endoscope system comprising:
the medical support device according to claim 1; and
an endoscope provided with the camera.

19. A medical support method comprising:
performing a measurement output process for measuring a size of an observation target region, based on a medical image obtained by imaging an imaging target region including the observation target region with a camera provided in an endoscope, the endoscope being inserted into a body cavity and for outputting the size; and
controlling the measurement output process in accordance with appearance information related to an appearance of a surface region in the medical image, the surface region being a portion of the imaging target region and including the observation target region.

20. A non-transitory computer-readable storage medium storing a program executable by a computer to execute a medical support process, the medical support process comprising:
performing a measurement output process for measuring a size of an observation target region, based on a medical image obtained by imaging an imaging target region including the observation target region with a camera provided in an endoscope, the endoscope being inserted into a body cavity and for outputting the size; and
controlling the measurement output process in accordance with appearance information related to an appearance of a surface region in the medical image, the surface region being a portion of the imaging target region and including the observation target region.
